(19) United States
(12) Patent Application Publication
(10) Pub. No.: US 2004/0176731 A1
(43) Pub. Date: Sep. 9, 2004

(54) Personal urine management system for humans

(76) Inventors: Gordon C. Cheng, Carlisle, MA (US); James R. Valentine, Reading, MA (US); Richard M. Beane, Minchan, MA (US); Daniel M. Beane, Hinghas, MA (US)

Correspondence Address: Kathleen Chapman, 765 Greenville Road, Mason, NH, 03048, US

(21) Appl. No.: 10/477,460

(22) PCT Filed: Oct. 31, 2001
(86) PCT No.: PCT/US01/45281

Related U.S. Application Data
(63) Continuation-in-part of application No. 09/826,778, filed on Apr. 5, 2001, now Pat. No. 6,569,133.
(60) Provisional application No. 60/195,040, filed on Apr. 6, 2000.

(51) Int. Cl. A61F 5/44

(52) U.S. Cl. 604/329

(57) ABSTRACT

A urine management system including components in fluid wicking communication with one another—a collection device, a conveyance tube, and a storage container—is disclosed. The gender-shaped collection device has wettable internal walls and contains a spacing wick. The male collection device additionally has a compression tube that provides a uniform compressive force for attachment and seal. The conveyance tube wicks urine from the collection device to the storage container, and contains a spacer throughout its length to prevent blockage by kinks or twists. The tube adapts to human shape and movement, expands during use, and contracts during idle periods. The storage container receives urine from the conveyance tube and immobilizes it. The storage container can be reusable or disposable. It's internal structure causes uniform distribution of the urine throughout the storage container. The urine system components provide continuous wicking through special connecting devices. The urine system components and special connecting devices may be serially connected to each other or to other types of components.

### BACKGROUND OF THE INVENTION

1. Field of the Invention

The present invention relates to the field of collecting urine and conveying it from point of collection to storage, and more particularly, to an improved urine collection, conveyance, and storage system for ambulatory incontinent humans.

2. Background Information

Systems for Urine Management

Urine-incontinent adult humans whose condition does not require use of an internal or “indwelling” catheter can either use a system of devices worn on the body to collect and to store the urine separately for periodic disposal, or can wear an absorbent pad or undergarment that both collects and stores the urine and that must be removed from the body for disposal (or cleaning) and replaced with an unused one periodically. A system of devices consists of a urine collection device, a urine conveyance device to transport collected urine to storage, and a urine storage container, generally a bag, that is most often worn attached to the user's leg. Such devices and the systems formed by their connection are the subject of this invention.

Self-contained urine management systems—collection, conveyance, and storage in one device—are constructed from multiple layers of absorbent materials. These self-contained systems, such as diapers, absorbent pads and absorbent undergarments, have several layers: a urine-accepting wettable layer next to the body, one or more absorbing layers, and a waterproof barrier layer outside of the absorbing layers that prevents urine from wetting outer clothing. The unitary structure of such absorbent systems in which the functional parts lie in contiguous, layered contact necessitates storage of the collected urine within or very close to the crotch, a body region that is not conducive for storing any significant quantities of urine. Accumulation of stored urine, which results in swelling and loss of rigidity of the absorbent structure, can result in discomfort for the user. State-of-the-art absorbent systems use a variety of super-absorbent polymer materials as well as mats or sheets of non-woven, wettable fibers to absorb the collected urine. Disposal of the collected urine is accomplished by removing the wetted product article from the body region, which is an action that often requires partial disrobing, donning a fresh article, and disposing of the entire wet article as sanitary waste.

Collection Devices

Urine collection devices for males are usually attached directly onto the penis; many are difficult to apply and to remove, and can lead to painful and potentially injurious penile skin conditions. Typical conveyance devices available are relatively thick-walled rubber or plastic tubing, which is stiff and bulky, and often visible under clothing. Leg-mounted storage bags are generally bulky and uncomfortable to wear, and, because of the drainage outlet, are potential sources of unexpected leakage. Urine management systems for females generally have fitted collection devices, with conveyance tubing and leg bags that are essentially similar to those used for males.

Male sheath catheters, or condom catheters, are typically very thin-walled, flexible tubes of latex or silicone rubber (about 0.003-inch in thickness) that are intended to enclose the penis in a watertight sack that is larger in diameter than the penis. They are usually attached to the penis using either a strip of double-sided adhesive tape, or an adhesive coating on a portion of the interior surface of the catheter. The adhesive is used to hold the catheter in place on the penis and to help form a liquid-tight seal between the catheter and the skin. The adhesive makes the application and removal of the catheter difficult and often painful to the user.

Male sheath catheters are dependent upon gravity for drainage. Incomplete drainage, or “pooling”, of urine can occur as a result of crimping of the catheter or catheter tube or when the user is in a seated position. Thus, a seated or prone user must rise to an upright position periodically in order to facilitate drainage or to relieve the pooling problem.

Female urine collectors are typically configured as cups, pouches, bags and cones, the larger of which are held in place by straps or a harness. A watertight seal typically of adhesive or foam, is formed between the user and the perimeter of the collector. Female collectors rely on gravity force for drainage.

Conveyance Devices

Conveyance devices to move urine from the point of collection to the storage device are typically narrow-bore tubes. The first priority in the design of a conveyance tube is to insure that it doesn't kink, become blocked, or collapse. Thick-walled tubing, sometimes with internal protrusions molded into the inner wall of the tubing, prevents kinking but can be rigid and uncomfortable to wear. Replacing thick-walled tubing with thin-walled tubing could result in tube collapse or sealing off of low volume flows such as urine incontinent leakage.

Storage Devices

The storage container is typically a flexible-wall reservoir that receives and accumulates liquid urine prior to discharge for disposal, and generally is either attached to the user's leg or suspended from the waist. These reservoirs are typically made from heavy-gauge sheeting of latex rubber, vinyl, or similar polymer with a single storage chamber. All liquid urine storage containers are provided with an outlet valve at a low point on the container to allow for draining. When used for successive fill-and-drain cycles, the containers must be cleaned and disinfected regularly to avoid odor and buildup of bacteria However, most urine containers are discarded after 1-2 weeks because they cannot be thoroughly cleaned.

In single-chamber flexible wall storage devices, collected urine accumulates at the bottom to give a localized bulge under clothing, which is visible and impedes movement of the wearer. The contained liquid tends to slosh around when the wearer moves creating noise and a disconcerting feeling. There are some prior art baffles or other internal attachments between the walls to reduce the wall bulging and the liquid sloshing; however the overall container still bulges. In addition, if the storage device becomes accidentally disconnected, the spill of contained liquid urine can be very messy. All prior art storage devices require venting of urine-displaced air, and thus, will emit an unpleasant odor.

Storage devices containing liquid-absorbent materials such as gel-forming polymeric absorbents are found in the prior art. In general, the prior art describes liquid absorption and gel formation in a single-chambered envelope or bag with an inlet and containing a gelling agent or absorbent either in a highly confined mass or in an undefined shape. No provisions are described for deliberate distribution or mixing of the fluid to ensure rapid wetting and gel formation, nor are any provisions made for ensuring a particular shape or form of the absorbed or gelled fluid other than noting the geometric shape of the envelope or bag.

Storage devices that take the form of leg bags rely on elasticized straps around the calf or suspension from a waist belt to maintain a desired position on the leg. Putting on these straps is tedious and time consuming. In addition, an improperly fastened leg bag may be more easily dislodged from the secure fastened position with consequent possible urine spillage.

One approach described in prior art to reduce localized bulging and liquid sloshing in storage devices is to immobilize the liquid by causing it to be imbibed into suitable absorbent materials that are present in the device in confined porous packages or within the device as unconfined solids in an undefined distribution This approach is subject to possible gel blocking that will result in unpredictable liquid uptake performance and under-utilization of absorbent. Gel blocking, which can occur in many absorbents that swell as water is imbibed, results from the formation and swelling of layers of partially or wholly-gelled liquid-saturated absorbent material, which layers significantly retard the transfer of free urine into the unused absorbent that lies beyond the gel layer.

“Blocked” absorbent materials take up additional urine at a slower rate than the dry materials. If the user of a urine storage device in which gel-blocking has occurred is able to discharge a strong stream of urine, the force of the urine jet may be sufficient to physically mix the absorbent to prevent blinding. On the other hand, a constant dribble flow of urine from a heavily incontinent person would not be sufficiently energetic to overcome the blinding, resulting in excessive free liquid in the device. Thus, prior art storage devices containing undistributed masses of absorbents, which are intended for healthy users, are not adequate for incontinent persons.

### SUMMARY OF THE INVENTION

This invention overcomes the disadvantages of the prior art by providing an improved urine management system and method of use for incontinent humans. The improved urine management system is formed by serial mechanical and continuous fluid connection of a gender-specific collection device, a conveyance tube, and a storage container. The improved system provides collection of incontinent urine leakage, even low-force trickle-type flow and residual drops and pools, from either males or females, and conveyance of the collected urine to the storage container that can be replaced periodically and/or disposed of Anti-gravitational flow of urine through the system is accommodated in the improved system.

Continuous fluid connection, in an illustrative embodiment of the improved system, is a result of the wicking of liquid urine. Wicking is defined as the flow of fluid through a matrix or bed of fine, interconnected capillary spaces. A wicking matrix will spontaneously imbibe a fluid that effectively wets the walls of those capillaries. Some components of the improved system, and the connections between the components, contain wicking elements that are placed in liquid-continuous connection to form a wicking flow path through the system. The continuous liquid flow path enables the removal of urine from a collection device through the system to a storage container that is separate from the collection device, even when the user is in a seated or prone position.

In contrast to existing ambulatory human urine management systems, the improved system is easy to put on, comfortable to wear, and convenient to maintain. It can be unnoticeable when worn under clothing and provides for a storage container that can be disposed in a sanitary fashion. The conveyance system preferably does not collapse nor become kinked or blocked, and the possibility of leakage can be reduced to a minimum. Liquid urine can be converted to an immobilized form and deliberately distributed throughout the storage container. The storage container may operate correctly without an outlet valve for draining.

The improved components can be used as a serially connected unit, or separately in combination with devices not described herein. For example, the storage container of the invention can be used with prior art collection devices and conveyance tubes.

Urine Collection Devices

Both male and female urine collection devices collect urine from the body and conduct urine either directly to a urine storage container that is positioned in a physically separated location, or to a conveyance device that conducts urine to a physically separate storage container. Neither collection device is intended for retention of any substantial volume of collected urine, and the presence of liquid urine in the collection device is considered as a transient condition that may occur while the urine is being conveyed to the physically separate storage container. Both male and female collection devices have three principal functional parts:

(a) An intake layer in the vicinity of the urine discharge location that receives and conducts urine to a urine transport medium;

(b) A wall layer, that may be either rigid or flexible, that lies outside of and encloses the side of the intake layer that opposes the urine discharge location side, and that can be either in sealed contact with the periphery of the intake layer or in sealed contact with itself, it can form a urine-impervious layer around the intake layer; the wall layer can have openings for forming urine-impervious sealed connection to the body surface surrounding the urine discharge location and for forming sealed connection to the urine conveyance device or to the urine storage container; and

(c) The urine transport medium located between the intake layer and the wall layer that receives urine from the intake layer and transports it away, illustratively by bulk liquid flow or wicking transport or a combination of both, from the intake layer to a point of connection with the storage container or conveyance tube.

The generic collector is the basis for three independent forms: one particularly suitable for human females, and two that are particularly suitable for human males; these latter two are differentiated by the terms sheath-type and pouch-type.

A sheath-type urine collector for human males collect and transfer emitted urine to a conveyance device for conveyance to storage. The collection device comprises a flexible conduction tube to confine emitted urine while that urine is being transferred to the conveyance device, an elastic compression tube to hold and to seal the conduction tube onto the penis, wicking components and wettable surfaces to aid urine transfer within the conduction tube, and a spacer component to prevent cutoff of the urine flow through the conduction tube due to crimping of the tube walls.

Within the conduction tube are an open-channel lumen for collecting and transferring free-flowing liquid urine to the conveyance device, and wicking and urine-wettable inner surfaces for collecting and transferring drops or pools of urine from within the region between the penis and the distal interior end of the collection device to the conveyance device. The conduction tube may also have within its lumen, a spacer (a separating element) to prevent the open channel from being completely closed off to fluid flow by kinks or crimps in the sidewall of the conduction tube.

In the pouch-type urine collector for human males, emitted urine is collected in a funnel-like article with a urine-impervious exterior and an interior made from a wicking material that can imbibe urine droplets even when the user is in a seated position. Emitted urine is transported out the collector into the conveyance tube through a connector and is subsequently discharged into urine storage devise. The perimeter of the pouch-type collector can be made to be pliable so as to provide a flexible barrier to urine leakage beyond the confines of the collector. The collector can be of several shapes so as to enable it to be held in place next to the human male body either by an article of clothing or by its own shape when equipped with fastening means to hold such shape, and does not require additional manual pressure to hold it in place.

In the collection device for human females, the body-contact surface of the urine-intake layer is wetted by urine thus enabling that surface to imbibe discharged urine. Below said body-contact surface, but still within the intake layer, there is a layer of a material that is hydrophobic and that also has small penetrations or openings that enable passage of urine from the body-contact side away from the body while inhibiting liquid passage in the reverse direction. The intake layer of the female collector is made from pliable materials that can conform to the shape of human females.

The perimeter of the collector body-contact surface is urine impermeable and also pliable so as to provide a flexible barrier to urine leakage beyond the confines of the collector. The collector can be of several shapes so as to enable it to be held in place next to the skin either by an article of clothing or by its own shape when equipped with fastening means to hold such shape, and does not require additional manual pressure to hold it in place in order to achieve urine collection that is substantially free from leakage.

The urine transport means may be merely a short extension of the collector body that is connected to the urine conveyance device, or it may be a longer tubing structure that is termed a “conduction tube” to differentiate it in location and connection from a tube-form conveyance device. This extension of the collector is constructed in the shape of, or in use becomes the shape of, a flat tube and contains a spacer to prevent the walls of that tube from becoming sufficiently crimped so as to prevent draining of urine. The spacer may also be constructed from the same or different material as the urine transport layer or may be a physical extension of the urine transport layer. The distal portion of the collector may be connected through mating fittings that enable the connection of wicking continuity to the conveyance tube, or directly to a storage device using the same type of wicking connection. In this latter case, the conveyance tube becomes an integral extension of the distal portion of the collector without need for any intermediate connecting fittings.

In addition to said urine wicking layer, the urine-transport means also contains one or more open channels that can act as transitory reservoirs for urine flows whose volume temporarily exceeds the capillary volume of the fluid transport means and that also can conduct such contained excess liquid urine to the point of connection with either a storage device or a conveyance tube.

The width, thickness, and material of the urine transport means are selected to accommodate the expected maximum urine discharge flow rate for a given application. For example, the application could be for receiving a sudden discharge from the partly filled bladder of an urge-incontinent user, or could be for receiving leaked urine from a completely incontinent person.

Urine Conveyance Devices

The conveyance tube connects and conveys urine from a collection device to a separately located urine storage container. Any tubing of any geometric shape that meets the objective of remaining flat or nearly flat when not in use can possibly be used as a conveyance tube. In an illustrative embodiment, the conveyance tube is a thin-walled polymer tube.

The conveyance tube can be constructed of a single or of multiple layers, at least one of which is urine-impervious, of thin materials, such as thin-walled plastic films, that are substantially conformable to human anatomical contours. In an illustrative embodiment, the thickness of the conveyance tube film layers may be in the range 0.0127 to 0.127 mm (0.0005 to 0.005 inch) and preferably in the range of 0.025 to 0.076 mm (0.001 to 0.003 inch). A conveyance tube can be made of multiple layers of thin films that are not bonded together. These layers can slide independently over one another thus retaining the flexibility of thin films while providing added mechanical protection for the urine-carrying tube elements.

The exterior circumference of the conveyance tube may be formed as a continuous enclosure as may be resulted from manufacturing methods such as extrusion or by blow molding or could formed by joining the edges or areas proximal to the edges of two separate stripes of the same material by welding or adhesive substances.

The “flat” property of such flat conveyance tubes can be characterized as having a thickness-to-width ratio (i.e., the ratio of the minor-to-major axes of the ellipse formed by the cross-section of the partially flattened tube) of from less than 1.0 to a low value limited only by the thickness of the spacer, and preferably in the range of 0.05 to 0.5. When flat, the illustrative embodiment of the conveyance tube has two exterior surfaces, and the tube may be positioned in such a way that one exterior surface lies against the skin of the user, the other surface away from the user. An outer layer may be added to the either or both exterior surfaces of the conveyance tube that is made from the same materials of the conveyance tube or from different materials to suit different purposes (e.g., both may be made from soft, breathable materials, one may have a fastening means such as a temporary adhesive for fastening the tube to skin or to clothing, etc).

A spacer is located throughout the length of the conveyance tube lumen. Each end of the conveyance tube can be fitted with suitable adapters to enable connection to suitable collection devices and storage containers. The spacer structure can have a range of thickness and width dimensions and stiffness that will prevent tubing closure while allowing the tube to flex and adapt to usage conditions and urine to flow unimpeded. The spacer can be independent of the tube walls, or can be attached to the tube walls. Examples of spacing material include woven materials (e.g., loosely knitted nylon, polyolefin, glass or metal fibers, textiles, etc), non-woven materials (e.g. rayon acetate needled felts, cellulose acetate fiber bundles, etc.), and flexible foams or solids (e.g., polyolefin film strips, polyolefin foam strips, silicone shapes, etc), depending on the specific application.

The spacer can wick urine. The spacer material can intrinsically wick or it can be treated to render it urine wettable, among other wicking spacer possibilities. Since wicking can promote the non-bulk flow of urine from one point to another within a fluid-continuous connected network, then making wicking-continuous connections of a wicking spacer in a conveyance tube to a urine collector and a urine storage device containing elements that are likewise wicking can enable the formation of a system for non-bulk flow conveyance of urine to storage.

In an illustrative embodiment, the conveyance tube can feed simultaneous connections to multiple storage containers.

A collapsible conveyance tube for conveying liquid, having an interior cavity, an interior surface defining the cavity, and walls, is disclosed comprising a means for spacing that is substantially removably disposed within the interior cavity. The means for spacing prevents complete collapse of the conveyance tube. The means for spacing comprises a material selected from a group consisting of loosely knitted nylon fibers, loosely knitted polyolefin fibers, flexible solid shapes of polyolefins, flexible solid shapes of silicone rubber, loosely knitted fiberglass, loosely knitted aluminum, polyolefin film, porous wicking materials including needled felts of rayon and cellulose acetate fiber bundles, natural woven fabrics, and synthetic woven fabrics. The walls comprise material selected from a group consisting of rubbery polymer such as silicone rubber, latex rubber, elastic or elasticized fabric coated, polyolefins, latex, and polymeric. The means for spacing is positioned within the interior cavity to prevent the conveyance tube from crimping sufficiently to block drainage of the urine and comprises a wicking material to enable fluid transfer through the capillaries of said wicking material. The interior surface is selected from a group consisting of wettable materials and non-wettable materials that have been subjected to surface treatments to render the interior surface wettable for holding liquid.

Urine Storage Devices

The storage device receives, immobilizes and stores urine that has been conveyed from a separate urine collector located at the region on human anatomical surface where urine discharge occurred. The storage device receives urine through a single inlet opening that is connected on to a urine conveyance tube or to a urine collection device. The urine storage device immobilizes received urine through one of more means of distributing the urine into predetermined locations within the device and rapidly converting the distributed urine to a form (solid, gelled-solid, or absorbed in capillary spaces) that no longer exhibits fluid properties. When the desired load of urine has accumulated in the storage device, the device can be detached from the connection with the collector or conveyance tube and, if desired, the connection opening on the device can be capped or covered. Subsequently, the used device along with the stored urine can be disposed of in a proper manner as a solid waste.

The storage device has four principal functional components: (a) an outer shell of a material that is impervious to urine liquid, that is sealed liquid-tight along all peripheral edges except for the inlet connector opening, and that is sized and configured to allow for expansion as needed when urine is being accumulated in storage; (b) a urine-distribution means that uses either one or a combination of gravity-driven and capillary-wicking fluid transport forces to distribute received urine more or less uniformly throughout the immobilizing matrix of one or more absorbents; (c) a urine-immobilizing absorbent means that, prior to contact with urine, is held in place mechanically and that may consist of a single kind or combinations of natural or synthetic fibers, cellulose fibers, water-absorbing natural or synthetic polymers or like materials in mats or in woven or non-woven sheets; (d) a single inlet connector that provides connections for both wicking continuity with and bulk fluid flow from the conveyance tube to the urine distribution system within the storage device.

The urine distribution means consists of a wick throughout its working length in one or more predetermined locations within the device and a fluid distribution channel formed between a barrier film and the wick. Transfer of the urine from the wicking material to the absorbent can take place in one of two ways: either by bulk flow or by a “bridging” transfer to the absorbent matrix brought about by close physical proximity of the wick to the matrix of absorbent or absorbents. The wick lie within or in contact with a barrier film that distributes urine to predetermined locations of absorbent matrix. Urine to be absorbed is conveyed by wicking, either alone or in combination with bulk flow of urine in fluid pathways adjoining the wick, into the immediate vicinity of unused or partially used absorbent material.

The matrix of immobilizing materials accommodates physical distribution of immobilizing materials and holds them in pre-determined locations within the storage device, and urine is distributed in a pre-determined manner so as to enable a more uniform volume expansion of the immobilizing materials as urine is absorbed. The matrix of immobilizing materials may be in a form of a physically mingled mass of non-woven fibers with one or more absorbents, or as one or more solid absorbent materials held in or on a physical structure such as a woven textile or non-woven mat of fibers, or as one or more absorbent materials coated onto the surface of the compartment wall in partially gelled form or by suitable attachment means, or as a material suitable for confining liquids by capillary force, or a combination of the above.

The external appearance of an unused storage device is of a thin, flattened form that expands more or less uniformly in thickness as it fills with urine. The storage device can be positioned, and that position maintained by any of a number suitable means (including the use of straps, adhesive tape, loop-and-hook fasteners, or in the pouch or pocket of apparel, among other ways), on several locations on the body of the human female user, e.g., on the calf or thigh in a wrap-around or substantially wrapped-around orientation, at the hip, etc. In addition, the device may be attached to the clothing of the user or may be attached to a nearby article, e.g. a wheelchair, bed, chair, etc. It can be worn attached to the user's body, e.g., worn on or around a leg, or attached to clothing. The storage device connects securely with a collection device directly or through a conveyance tube, and so is suitable for an ambulatory incontinent human when attached to the leg or waist or when attached to clothing at suitable points, and by a non-ambulatory human when attached to a suitable location in his/her immediate vicinity.

The storage device may be constructed so as to exclude entrapped air, thus enabling an unused device to have a volume somewhat less than that of an unused device that contained entrapped air. The ability of the storage device to exclude entrapped air also avoids the need to vent air that is displaced when urine is admitted into the storage device. The storage device is disposable.

Systems of Devices for Collection, Conveyance, and Storage of Urine

A novel system comprised entirely of said novel devices can be formed by use of connection means that are part of the novel devices to make serial mechanical and fluid connections between a novel collection device, a novel conveyance device, and a novel storage device. The connections formed by the connection means that are part of the novel devices enable formation of fluid-continuous wicking connection of the wicking fluid transfer capabilities in each of the novel devices, thus enabling the operation of the system for continuous wicking conveyance of urine.

The ability of the novel system to wick small amounts of excess liquid urine, whether remaining from a drawn-out voiding or resulting from a nearly continuous dribbling stream experienced by a completely incontinent user, provides an extra clear benefit. System performance is more than the performance of the individual devices when they connected in combination with prior art devices.

A mixed system containing at least one of the novel devices and at least one of the corresponding devices from prior art can be formed by using one or more connection devices from a set of novel connection devices. These novel connection devices enable formation of connections of said novel devices for collection, conveyance, and storage of urine to prior art devices in such a manner that the capabilities of the novel devices in the mixed system are not interfered with or otherwise rendered inoperative such as would occur by use of other more common prior art connectors. In such a mixed system, the improved capabilities of the novel devices present will enhance the overall performance of the mixed system with respect to the prior art systems. However, said mixed systems will not have all of the capabilities of the novel system described above.

It is also entirely within the scope of the instant invention that the devices and systems for urine management disclosed can be used to provide urine management means for non-incontinent humans. For example, such systems and devices can be of benefit to humans in situations where access to a toilet for normal urine voiding is not possible or where such access is undesirable because of time delays and/or effort required to leave their situation, use the toilet to void normally, and then re-enter their situation.

Formation of Improved Devices and Systems from Prior Art Devices

It is entirely within the scope of the instant invention to include the modification of prior art devices for collection, conveyance, and storage of urine and systems comprised from such devices so as to provide in them capabilities for wicking transport of urine and for prevention of tube blockage. It is also entirely within the scope of the instant invention to include the modification of prior art absorbent systems, including such product categories as diapers and absorbent pads, by addition of a urine-impervious layer that lies over the urine-wettable layer and is joined over its perimeter in sealed contact with the corresponding water-impervious outer layer except for the opening intended for conveyance of urine into the storage article.

Means for providing said capabilities includes placement of one or more physical structures within the lumen of prior art devices to prevent the complete closure of thin-walled tubes or to provide all or part of a pathway for the wicking conveyance of urine, and application of chemical or physical treatments to the interior lumen walls of prior art devices so as to form one or a multiplicity of continuous urine-wicking pathways on those lumen walls. Formation of wicking connections as described in the instant invention will enable the formation of novel systems by serial connection of said improved devices that have been formed from prior art devices.

### BRIEF DESCRIPTION OF THE DRAWINGS

The above and further advantages of the invention may be better understood by referring to the following description in conjunction with the accompanying drawings in which like reference numerals indicate identical or functionally similar elements:

FIG. 1 is a semi-transparent, diagrammatic view of the major parts of an illustrative embodiment of the invention;

FIGS. 2A and 2B are the external and cross-sectional views, respectively, of an illustrative embodiment of the sheath-type male urine collector;

FIGS. 3A, 3B, and 3C are the external, cross-sectional, and top views, respectively, of an illustrative embodiment of the pouch-type male urine collector;

FIGS. 4A and 4B are the body contact surface and opposing surface, respectively, of an illustrative embodiment of the female urine collector;

FIGS. 4C, 4D and 4E are front-standing, side-standing, and seated views of illustrative embodiments of the female urine collectors as worn on the body;

FIGS. 5A and 5B are cross-sectional views of illustrative embodiments of the female urine collector;

FIG. 5C is a cross-sectional view of an illustrative embodiment of the body contact surface of the female collector;

FIG. 6A is a transparent view of an illustrative embodiment of the conveyance tube;

FIG. 6B is a cross-sectional view of an illustrative embodiment of the conveyance tube;

FIGS. 7A-B are exploded perspective views of an illustrative embodiment of the layers of the absorbent matrix type urine storage device;

FIGS. 7C-D are a front view and a side cross-sectional view, respectively, of an illustrative embodiment of the absorbent matrix type urine storage device;

FIG. 7D shows examples of the distribution means of absorbent matrix type urine storage device;

FIG. 8 shows the cross-sectional view of an illustrative embodiment of the absorbent matrix type urine storage device;

FIG. 9A is an exploded perspective view of an illustrative embodiment of the compartmental type urine storage device;

FIGS. 9B and 9C are views of perforated and solid compartment layers, respectively, of an illustrative embodiment of the compartmental type urine storage device;

FIG. 10 is a transparent front view of an illustrative embodiment of the compartmental type urine storage device;

FIG. 11 is a cross-sectional side view of an illustrative embodiment of the compartmental type urine storage device.

FIGS. 12A, 12B, 12C, and 12D are semi-transparent views of connection and continuous fluid path mechanisms used in collection-conveyance and conveyance-storage connections of an illustrative embodiment of the invention and in the prior art, respectively;

FIGS. 13A, 13B, and 13C are, respectively, transparent views of illustrative embodiments of a continuous fluid path wicking flow system in upgradient flow of urine over a high point, in upgradient flow to a storage point higher than the urine collection location, and in pool and drop removal from within interior regions of the collection device and conveyance tube;

FIG. 14 is a transparent view of an illustrative embodiment of a multi-output conveyance tube connected to multiple storage containers;

FIGS. 15A and 15B are transparent views of illustrative embodiments of the sheath-type male urine collector in which the conduction tube is attached to the compression tube and the conduction tube underlies the compression tube, respectively;

FIGS. 16A and 16B are top/side and perspective views, respectively, of an illustrative embodiment of the spreading tool engaged in the pockets of the male collection device compression tube prior to opening the device for use;

FIGS. 16C and 17 are top and perspective views of illustrative embodiments engaged in the pockets of the male collection device compression tube after opening the device for use;

FIG. 16D are perspective views of three illustrative embodiments of the spreading tool;

FIGS. 16E and 16F are perspective views of a fourth illustrative embodiment of the spreading tool alone and in use, respectively;

FIGS. 18A and 18B are perspective views of an illustrative embodiment of the sheath catheter with velcro or adhesive enclosure, open and closed respectively;

FIGS. 19A and 19B are perspective views of an illustrative embodiment of the sheath catheter with zipper enclosure, open and closed respectively;

FIGS. 20A-C are perspective views of an illustrative embodiment of the sheath catheter with elastic tubing for holding the catheter in place;

FIGS. 21A-E are perspective views of an illustrative embodiment of the sheath catheter with rigid plastic bellows for holding the catheter in place;

FIGS. 22A-G are perspective views of an illustrative embodiment of the sheath catheter with a spring coil ring for holding the catheter in place; and

FIGS. 23A-C are perspective views of an illustrative embodiment of the sheath catheter with hand-held jaw ring expander for extending the sheath over the penis.

### DETAILED DESCRIPTION OF AN ILLUSTRATIVE EMBODIMENT

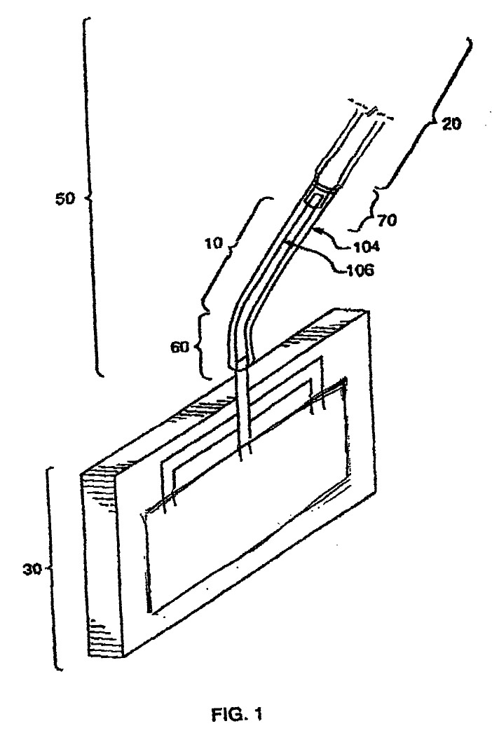

Referring to FIG. 1, the novel urine management system 50 comprises urine collection device 20 for human that collects and transfers urine to a urine conveyance tube 10, which conveys urine to a urine storage device 30 wherein the urine is converted to and stored in immobilized form prior to disposal. All three components of system 50 are serially connected as in FIG. 1, i.e. urine collection device 20 is connected to urine conveyance tube 10 through connection mechanism 70, and urine conveyance tube 10 is connected to urine storage device 30 through connection mechanism 60. Connection mechanisms 60 and 70 between devices 10, 20, and 30, allows gravitational drainage of liquid and, with wicking transport means within each device, enables the creation of a continuous path of wicking transport to conduct urine from the collector to the storage device.

In simplest form, connection of these novel components as urine management system 50 provides continuous collection, conveyance, and storage of liquid urine from incontinence leakage into novel urine collection device 20, conveyance of that urine through novel conveyance tube 10 flowing under gravitational influence, and absorption and storage of the urine in immobilized form in novel urine storage device 30. When storage device 30 has become sufficiently filled with urine, it is replaced with a fresh unfilled storage device, and said urine-containing storage device is disposed of as solid sanitary waste.

Devices 10, 20, and 30 are independent components, and can be disassembled and reassembled replacing only those used or spent components that require replacement for proper functioning of the system. Under most conditions of use of the urine collector that require wicking to convey the collected urine, it is necessary to use a conveyance tube with wicking capability such as described in this invention In certain instances, where the user is heavily incontinent, thus having a urine flow profile of frequent small volumes, and where the user remains in an upright (standing) position, it may be possible for the person to use a conventional rubber or polymer connecting tube and a conventional storage or leg bag. In such cases, connection of the collector 20 to conventional rubber tubing and a conventional storage bag can be accomplished by means of special connecting devices to replace connecting devices 60 and 70.

Each of the devices contains a wicking spacer component throughout the working length of the lumen of the device—wicking spacer elements 260 in the collection device, conveyance tube wicking spacer 106 in the conveyance device, and storage wicking spacer 340 in the storage device. When devices 10, 20, and 30 are serially connected as shown in FIG. 1, then the respective wicking spacers are brought into contiguous contact by the coupling of connectors 290 and 11, and by coupling of connectors 12 and 341.

Male Urine Collectors

The novel urine collectors for males, both the sheath-type, referring to 2A and 2B and the pouch-type, referring to 3A and 3B, collect and transfer urine from penis 261 to a novel urine conveyance tube 10 that then conveys said urine to a novel urine storage device.

Sheath-Type Male Urine Collectors

Referring now to FIGS. 15A and 15B, the sheath-type urine collection device 20 comprises a thin-wall conduction tube 250 of diameter sufficient to surround but not constrict penis head 262 as well as a wicking element 260, and an elastic compression tube 210, which can be attached to conduction tube 250, as illustrated in FIG. 15A, or can overlie said conduction tube 250, as illustrated in FIG. 15B. Conduction tube 250 has a connection 290 at its distal end for connection to a urine conveyance tube. Conduction tube 250 may also contain spacer or wicking elements 260.

In FIG. 15A, the conduction tube is attached to the compression tube 210, which is sized to provide an area of radial compression contact on the penis shaft that is sufficient to minimize the slippage of said tube 210 from its location on the penis skin surface, and to seal against leakage of urine from the device. In the “attached” embodiment, illustrated in FIG. 15A, conduction tube 250 is connected to elastic compression tube 210 via mutual connection to elastic interface 251, said connections being made while elastic compression tube 210 is in a radially-stretched condition in order to ensure a leak-free connection when tube 210 is in its stretched mode on a penis. Connection to interface 251 can be made by joining methods suitable for materials that change dimension during use, such as bonding with elastomeric adhesives, or other techniques known to those skilled in the art. If desired, portions of the skin contact surface of elastic compression tube 210 can be treated to provide additional frictional (“anti-slip”) characteristics or to provide a barrier against urine leakage.

In the embodiment shown in FIG. 15B, the conduction tube underlies the compression tube 210, both are sized and constructed to prevent slippage and leakage. In this embodiment, conduction tube 250 is extended proximally along the shaft of penis 261 a distance sufficient such that compression tube 210 is completely underlain by a portion of conduction tube 250. Compression tube 210 is located at a position on said penis so as to provide compression onto conduction tube 250 and thence onto the underlying portion of said penis.

Conduction tube 250 can be made from a rubbery polymer such as silicone rubber or latex rubber, from poly-olefin or other flexible film materials, or from fabrics, including elastic and elasticized fabrics, that can be coated or treated if needed to minimize movement of said tube on the penis skin and the leakage of liquid or vapors. The wall thickness is selected to provide the physical flexibility and conformability to facilitate liquid sealing and frictional stability on the skin surface.

Elastic compression tube 210 may be made of one or more layers of woven elastomeric fabric material, non-woven elastic fabric, elastic fiber-containing fabric, or elastomeric sheeting made from latex or silicone rubber. If a fabric material is used, appropriate areas of said fabric can be treated with water-repellant materials such as silicone oils to prevent liquid urine from penetrating the fabric.

Referring now to FIGS. 15, 16, and 17, in order to fit compression tube 210 over penis 261, when said penis is in the flaccid state, it is necessary to enlarge the area of the lumen of elastic compression tube 210 sufficiently. Said enlargement action is required both for the attached embodiment illustrated in FIG. 15A and for the overlying embodiment illustrated in FIG. 15B. Said enlargement action can be accomplished by applying outward tension forces onto three or more tensioning points which are more or less uniformly spaced around the circumferential perimeter of the opening of tube 210 that is intended for insertion of the penis.

FIG. 16A illustrates the enlargement of the lumen of the compression tube 2101 using spreading tools 280; the direction of movement of parts and components is indicated by small arrows enclosed within parenthetical marks (→). Tensioning points 2141 are formed when the tips 281 of tube-spreading tool 280 are inserted into receiving openings 2121, which are located on the perimeter of tube 2101, while the legs of said tool are in compressed state 282, as shown in FIG. 16B. The lumen is enlarged by applying outward tension to four tensioning points 2141 located equal-distance on the circumference of the compression tube 2101. As illustration in FIG. 17, Said tensioning forces are applied to an extent necessary to increase the dimensional size of the rectangularly-shaped opening of tube 280 so as to allow insertion of a penis 261.

It should be understood that other means exist for providing expansion forces for the tube-spreading tools 280, such as making use of spring-material spreaders or pivoting arm or such mechanical means familiar to those knowledgeable in the art.

The key actions of this aspect of the instant invention are: (1) the temporary enlargement of the opening of tube 210 by mechanical stretching so as to allow passage of a penis 261 into the lumen of expanded tube 210, and (2) subsequent release of the expansion forces so as to allow tube 210 to radially compress said penis. Such stretching can be accomplished by many types of mechanical devices familiar to those skilled in the art and the examples cited are not meant to limit the means used.

Now, referring to FIGS. 1, 2, and 15A-B, in an illustrative embodiment for novel urine collection device 20, a wicking element 260 is located within conduction tube 250 between penis tip 262 and collector-conveyance connector 290. Said wicking element 260 acts as wicking flow channels for liquid urine and also serve to prevent blockage of flow of free liquid urine when conduction tube 250 become crimped. The portions of wicking element near to the tip of penis 260 may be split to enable effective contact with the inner surface of tube 250. All of or selected areas of the interior surface of conduction tube 250 are rendered wettable by urine by any one of several methods known to those skilled in the art. When said areas of interior walls of tube 250 that are rendered urine-wettable are in contiguous contact with wicking elements 260, wicking transfer of urine between wall and spacer element is enabled. Thus, any drops or pools of urine that contact the urine-wettable interior wall areas of conduction tube 250 will be caused to wet and spread out on said wettable surfaces until the spreading liquid urine makes contact with wicking elements 260 which will wick up said urine, removing it from the wall and conveying it to connector 290.

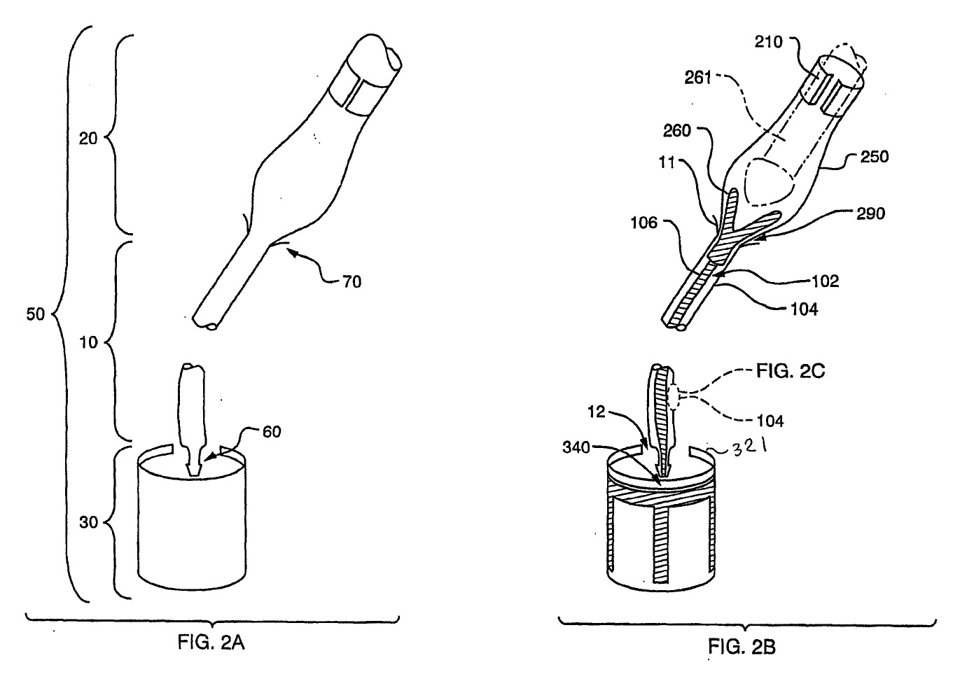

In order to enable effective connection of novel urine collection device 20 to existent art tubes for conveying urine, and referring now to FIGS. 2, 15A, and 12C, connection 290 attached to thin-walled conduction tube 250 can be connected through adapter fitting 24 that will cause wicking connections 260 and 23 to be located in a region within the connection that will not interfere with gravitational flow of urine into said connected existent art urine tube 10EX. It should be clear that addition of wicking materials to such prior art sheath catheters, none of which are currently equipped for such novel wicking transport, will result in conversion of such catheter devices to fall within the description of the novel conduction tube devices for collection of urine of the instant invention

Pouch-Type Male Urine Collector

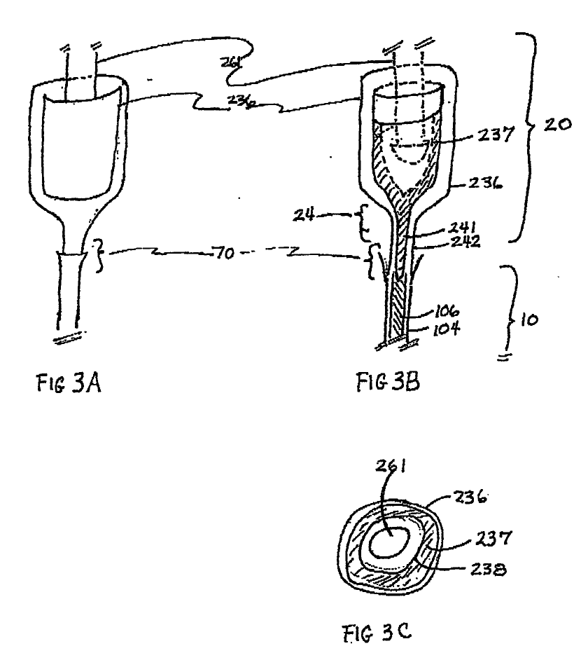

Referring now to FIGS. 3A and 3B, the pouch-type male urine collection device 20 is formed by shaping a wicking material 237 into a funnel-like configuration which may or may not have an intact perimeter. The larger opening can accommodate a penis and the small opening leads to connection means 24, which connects to conveyance tube 10 or directly to urine storage device 30. The overall wall structure 235 consists of an outer urine-impervious layer 236, which encloses the wicking material 237 to prevent urine leakage through the wall, and an inner lining material 238 that lies next to penis 261. The wicking material 237 along with the inner lining material 238 forms a lumen sufficiently large to encircle at least the distal end of the penis. The lumen of the wicking material 237 diminishes in cross-section near the small opening of the urine collector and eventually collapses into a single element 241. In a like manner, the outer urine impervious layer 236 transitions to become the outer wall 242 of the single element 241. In another word, the overall wall structure 235 transitions from the large opening to the small opening and becomes extension 22, which connects to the conveyance tube 10 through connector 70. The overall wall structure is constructed of flexible materials such that the wall structure 235 can conform to body contour of the user. The pouch-type male urine collector transports urine by capillary wicking flow through wicking materials 237, supplemented as needed by gravity. The perimeter of the pouch-type collector can be made to be pliable so as to provide a flexible barrier to urine leakage beyond the confines of the collector. The collector can be of several shapes so as to enable it to be held in place next to the human male body either by an article of clothing or by its own shape when equipped with fastening means to hold such shape, and does not require additional manual pressure to hold it in place in order to achieve urine collection that is substantially free from leakage.

Female Urine Collector

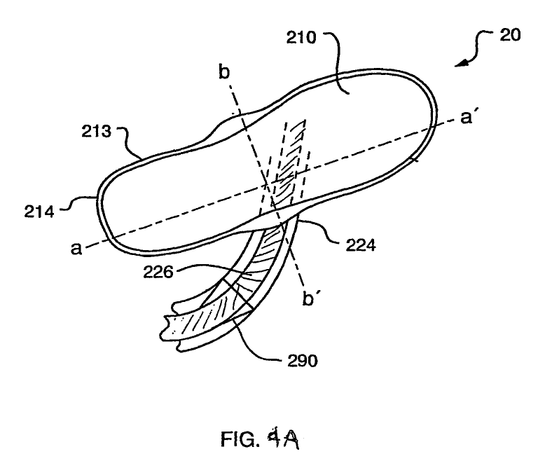

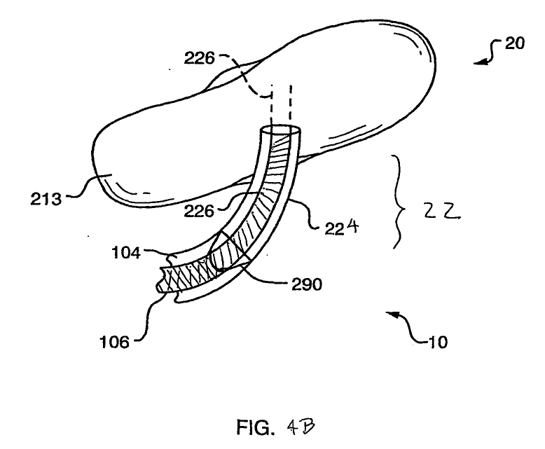

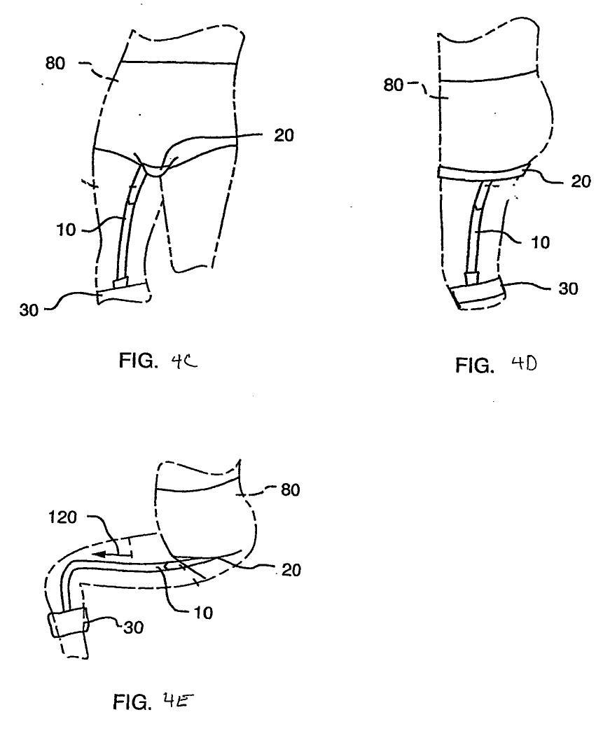

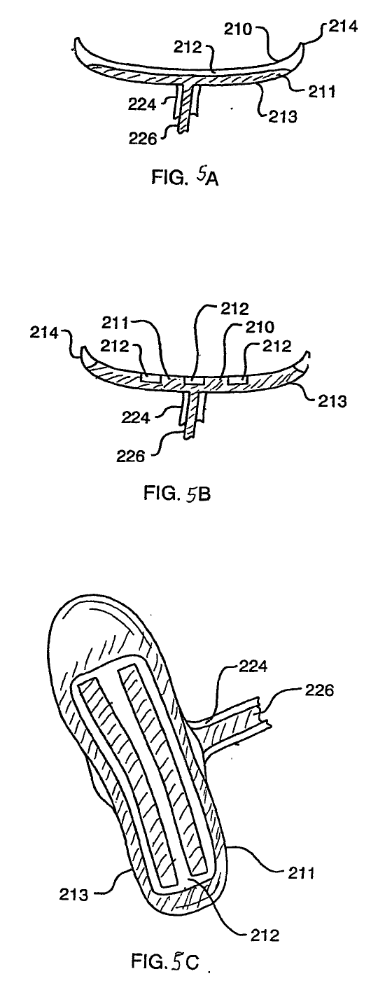

Referring to FIGS. 4A, 4B, 5A, 5B, and 5C, female urine collector 20 comprises a thin-wall flexible structure 21 for collecting urine from the wearer and extension 22 that incorporates spacer 226 to conduct urine from within structure 21 to collector-conveyance connector 290. Flexible structure 21 is comprised of a skin-contact surface 210 with underlying regions of wicking materials 211 that may incorporate interspersed and possibly interconnected open channels 212 that directly contact the underside of skin-contact surface 210 and that are also in contact, on one or more walls, with the wicking materials 211. Collector flexible structure 21 has a water-impermeable liquid containment layer 213 located either on its outer surface, or between its outer surface and the active urine collection and transport region containing the wicking materials 211. Said water impermeable layer serves to prevent leakage of urine through the outer layers of the collection device. Transport of urine is accomplished via capillary wicking flow through wicking materials 211, supplemented as needed by gravity flow, or transitory retention, of excess urine in open channels 212 or in wicking materials 211. While FIGS. 5B and 5C show three open channels 212, it should be understood that such channels may be present in any number, or may be absent, depending upon the volume-time profile for urine flow that the particular collector is intended to accommodate.

Referring to FIGS. 4A, 5A and 5B, skin-contact surface 210 is formed from one or more layers of urine-permeable materials that allow easy passage of urine to underlying wicking material 211 and open channels 212. Surface 210 is sufficiently pliant so that it will conform to the anatomical surface contours of the wearer's body to an extent sufficient to enable removal, by capillary pickup and wicking action, of droplets or films of liquid urine that may be transferred to one or another of the skin surfaces in the anatomical region during a urine voiding or leakage incident. Urine-impermeable barrier 214 on the perimeter of structure 21 is comprised of one or more pliant urine-impermeable materials that will conform to the contours of the skin so as to provide a barrier to the leakage of urine out of the collector region before all of the urine can be transferred through contact surface 210. Urine-impermeable barrier 214 may also be formed by coating the outer edge of structure 21 with a water-impermeable coating that will penetrate the depth of contact surface 210 at the perimeter, thus blocking outward wicking flow within structure 21, and that can also be sufficiently physically stable so as to permit formation of physical structure above contact surface 210 at barrier 214.

FIG. 4B shows the exterior surface, opposite the body-contact surface, of collector structure 21. Thin-wall extension 22 is attached to the liquid containment layer 213 to assure a waterproof seal, such as adhesive or heat bonding. Spacer 226 is continuous, in an illustrative embodiment, under liquid containment layer 213 in contact with the adjacent wicking materials 211 in order to assure a good wicking path. However, it should be understood that such wicking connections could be accomplished by interposition of added pieces of wicking materials in contiguous, fluid-transfer contact with each other.

In addition to providing wicking conduction of urine within thin-wall extension 22, spacer element 226 also serves to prevent complete closure of the lumen of extension 22 and resulting blockage of urine flow by crimping actions such as might occur from leg movement or from entrapment under elasticized edges of undergarments 80. This action and role of spacer 226 is analogous to that of spacer 106 in conveyance tube 10.

In an illustrative embodiment, conveyance tube 10 and collector extension 22 both comprise waterproof tube film layers 104 and 224, respectively, that can easily conform to varied and changing contours and shapes, and that can be made from thin-walled plastic film, e.g. 2-mil polyethylene

Referring to FIGS. 4C, 4D and 4E, the female urine collection device 20, held in position by undergarment 80, is connected to urine storage device 30 via flexible, thin-wall flat conveyance tube 10. Urine storage device 30 is initially in a flattened state. Urine management system 50 allows for collection of urine leakage into urine collection device 20, conveying that urine through conveyance tube 10 flowing under wicking action and gravity flow, and then absorbing and storing the urine in immobilized form in urine storage device 30 for ultimate disposal of the spent device as solid waste.

Conveyance Tube

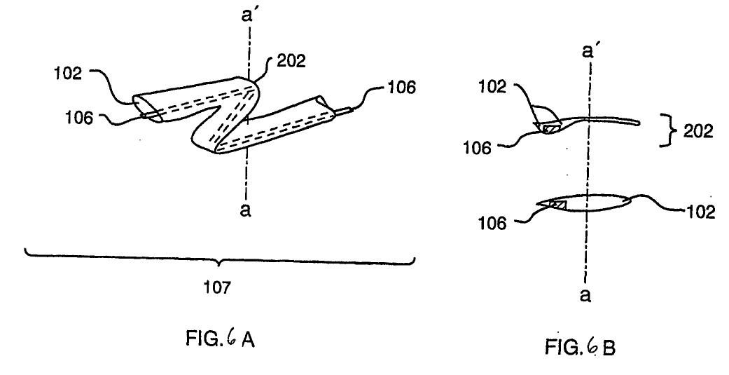

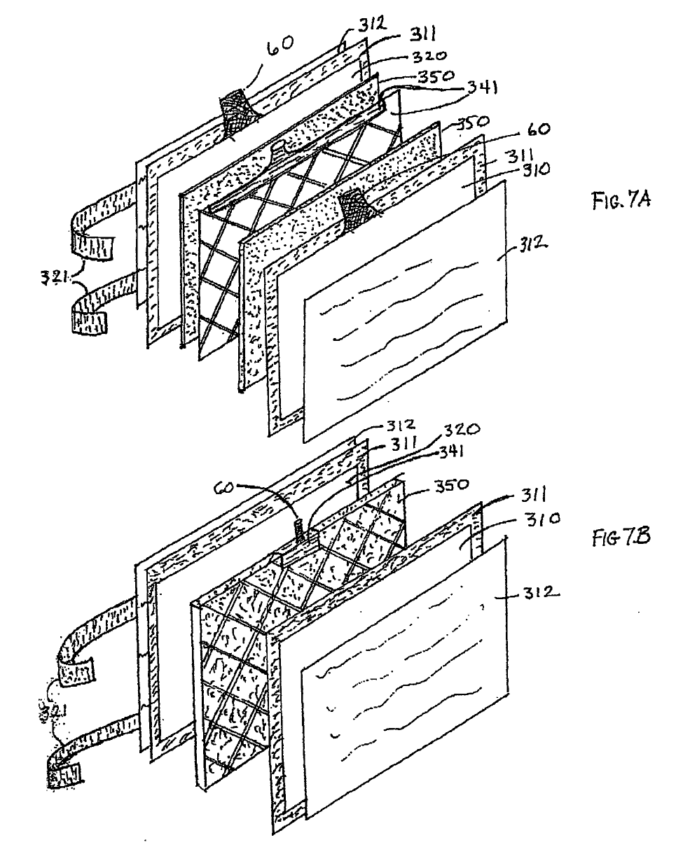

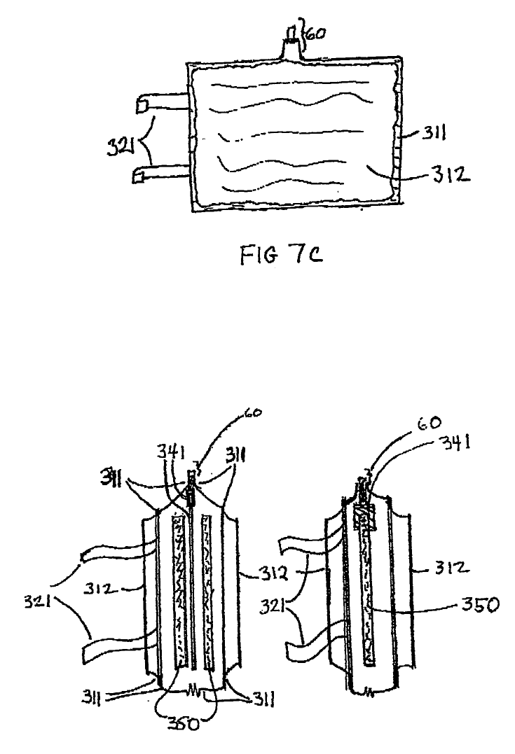

Referring now to FIG. 6A, conveyance tube spacer 106 spans continuously along the length of the lumen of conveyance tube 10 to prevent tube 10 from becoming closed to urine flow when opposite sides of wall 104 are pressed together by an external force in a crimped or kinked position 202. Conveyance tube spacer 106 prevents complete closure of internal wall surfaces of tube wall 104 leaving enough tube opening area 102 to allow the expected fluid flow to pass. Conveyance tube spacer 106, which may have the same or a variety of different cross-sectional shapes along that length, is constructed of a material that may or may not wick urine, and that is flexible to allow conformance to anatomical contours, has an open, porous internal structure or a high external surface roughness that will result in a porous leaky seal even when pressed against the internal surfaces of tube 10, and is relatively more wettable by urine than the tube-wall material, and to flow within the interstices of spacer 106. Conveyance tube spacer 106 may be made in several forms and from a variety of materials: from aggregates of fibrous materials that derive their physical stability from the aggregation (for example, single component or blended fibers of wool, cotton, rayon, nylon, polyester, etc, in the forms of yarns, woven fabrics, mats or felts); from open-cell foamed polymers and elastomeric that are wetted by aqueous fluids and that derive their physical stability from the polymer network (for example, polyurethane foams); from open-mesh materials that derive their physical stability from the strength of the individual bound fibers (for example, fibrous mats or masses, meshes and “fiber pads” of synthetic polymers such as polypropylene, and nylon); or from flexible solids (for example, rubbery polymers such as latex and silicone rubbers).

In an illustrative embodiment, conveyance tube spacer 106 is made of a material with wicking properties, is capable of conveying and guiding the flow of urine that wet those materials and fill the capillary spaces between the fibers or walls of the foam cell, and has sufficient physical strength to resist compression by an external force. Thus, in one embodiment, conveyance tube spacer 106 is constructed of rayon felts with a width from approximately 15 to 50 mm (0.6 to 2 inch) and a thickness from approximately 2.54 to 5.08 mm (0.1 to 0.2 inch), while in another embodiment, spacer 106 is constructed of bonded cellulose acetate fiber bundles. Referring now to FIGS. 4E and 6A, conveyance tube spacer 106 allows fluid to be wicked upgradient 120 to an anatomical region which is higher than urine collection device 20, from which point the fluid can then flow down to storage device 30 which is at a point that is lower than urine collection device 20, thus forming a gravity-driven siphon flow arrangement. Spacer 106 provides the wicking pathway, even when tube 10 is kinked 202.

Urine Storage Device—Absorbent Matrix Type

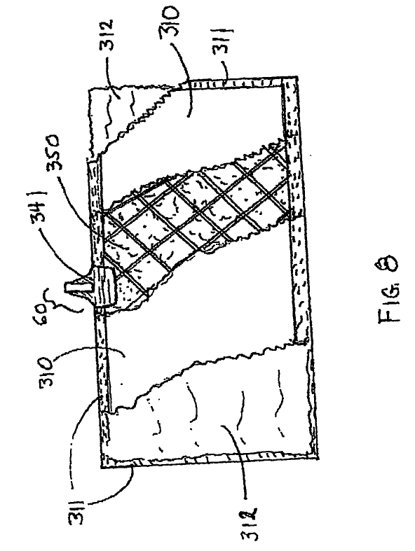

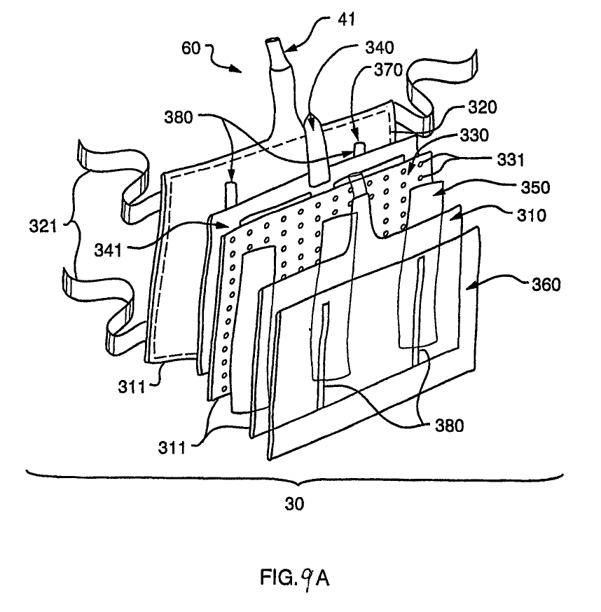

Referring now to FIG. 7A, a urine storage device 30 of the absorbent matrix type comprises at least one each of urine distributor 341 and absorbent matrix 350, all in contiguous fluid contact when liquid is introduced into the storage device via storage devise connector 60 which connects with conveyance device 10. Components 341 and 350 are contained within a structure formed by sealing a liquid-impervious back wall 320 and liquid-impervious front wall 310 along their entire peripheral edges 311 except in the vicinity of conveyance tube-storage device connector 60. Peripheral edges 311 are sealed using adhesive or heat-sealing bonding techniques that are well known in the art.

Referring to FIG. 7D, the urine distributor 341 maybe configured in a number of ways to provide more or less uniformly distribution of incoming urine horizontally with respect to gravity so as to avoid localized liquid absorption that prevents complete utilization of the absorbent materials and causes unequal weight distribution in the storage devise. The urine distributor 341 consists of a wicking material that makes contact with one or both side of the absorbent matrix The urine distributor 341 may be sized to contact a portion or the entire absorbent matrix. The distribution function can be aided by forming channels or patterns directly on the surfaces of the absorbent matrix. The urine distributor can be made from a number of materials that possess wicking property. Absorbent matrix 350 used in the storage device of the matrix type may be formed by distributing active absorbent within a mat of water wettable, non-water wettable fibers and other fibers, such as, thermally-bondable polyolefins, to aid in stabilizing the physical structure of the matrix. Examples of such absorbent matrix are superabsorbent composites commonly used in urinary incontinence and feminine hygiene products.

Urine Storage Device—Compartmental Type

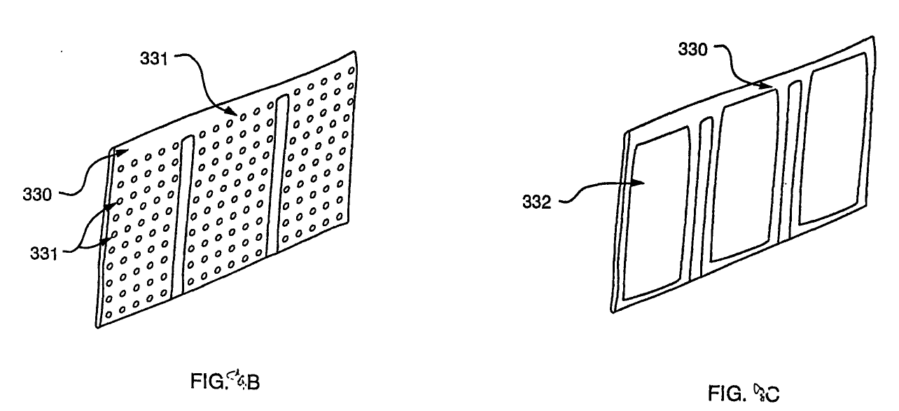

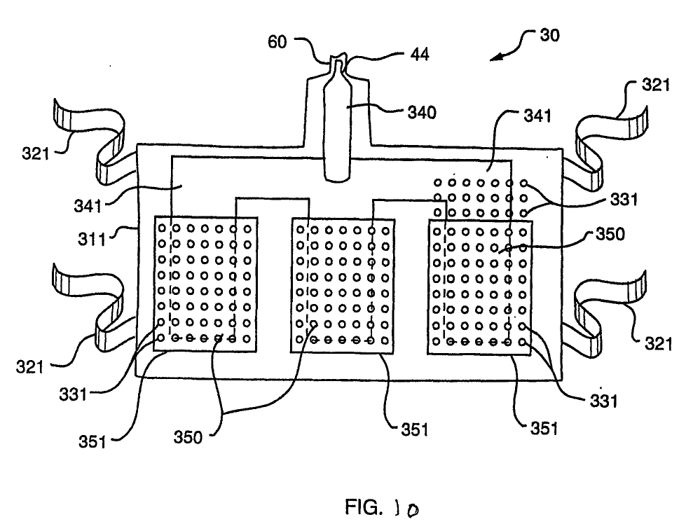

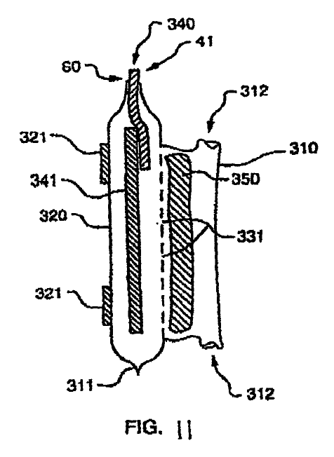

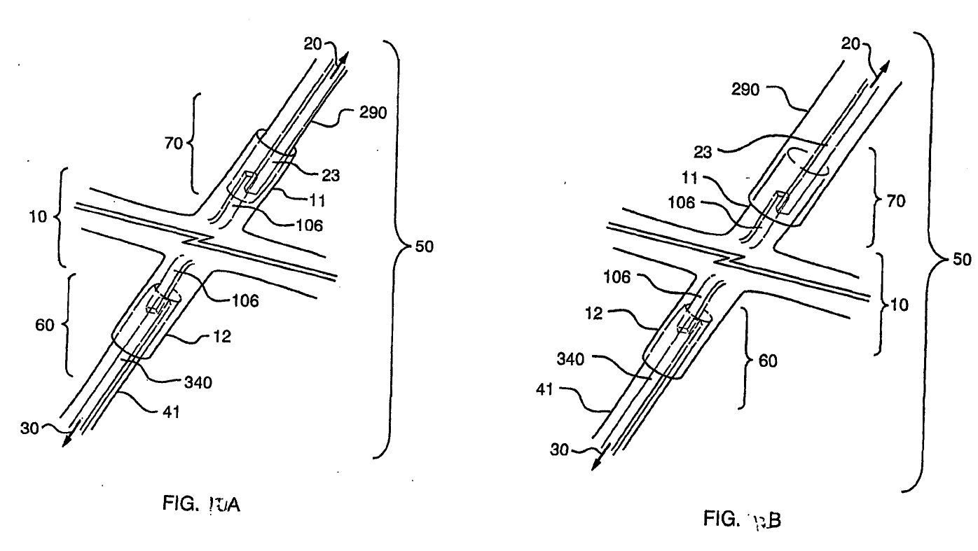

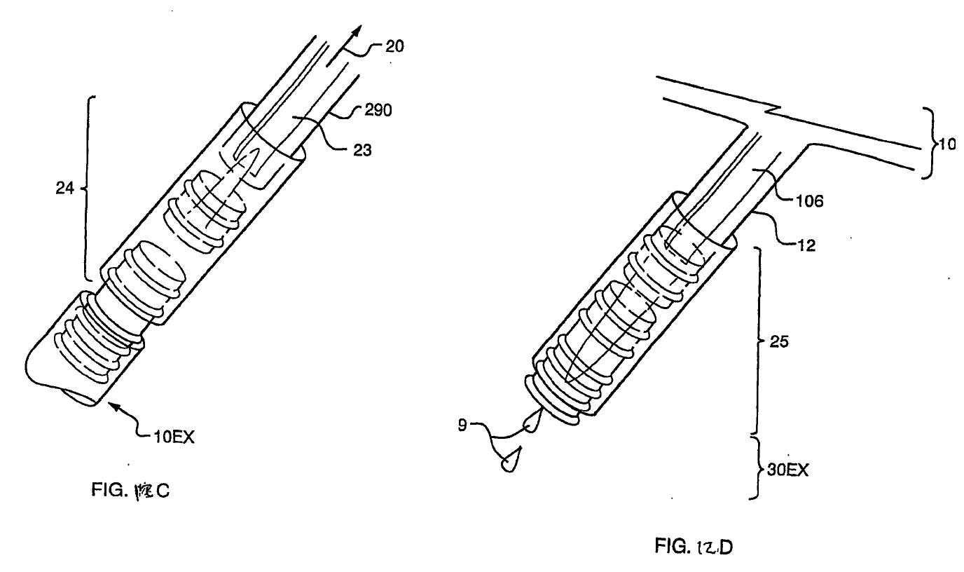

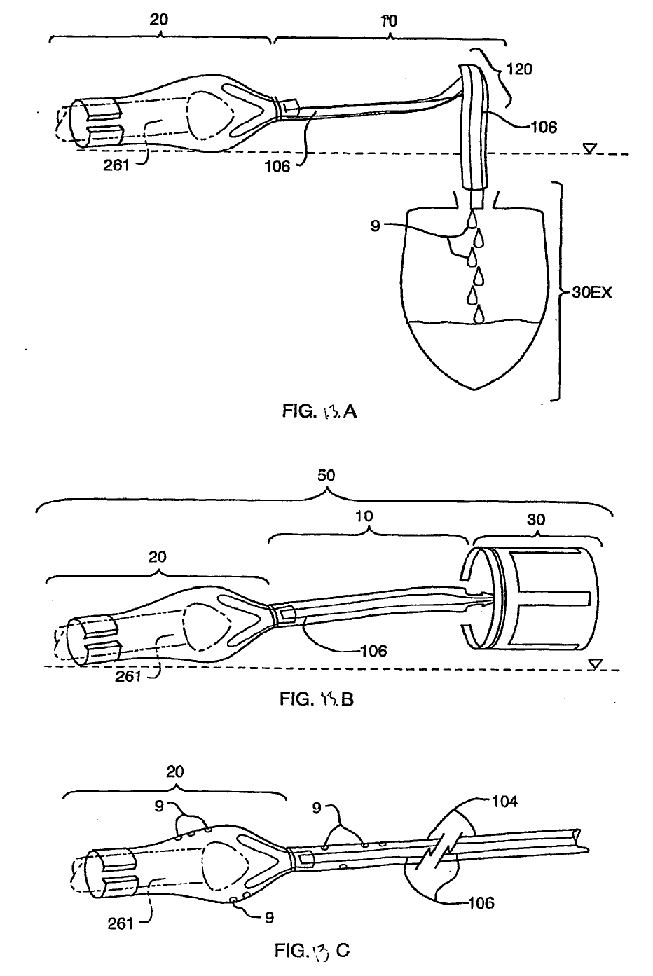

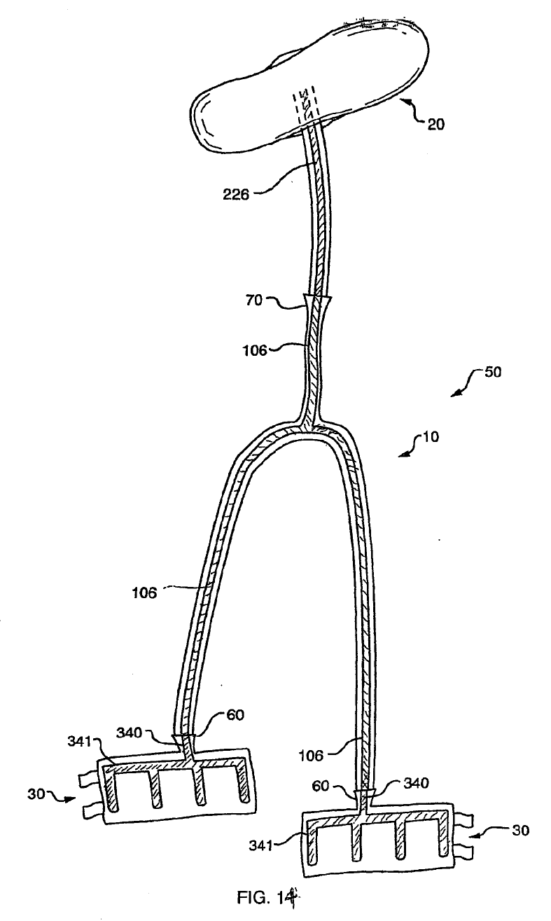

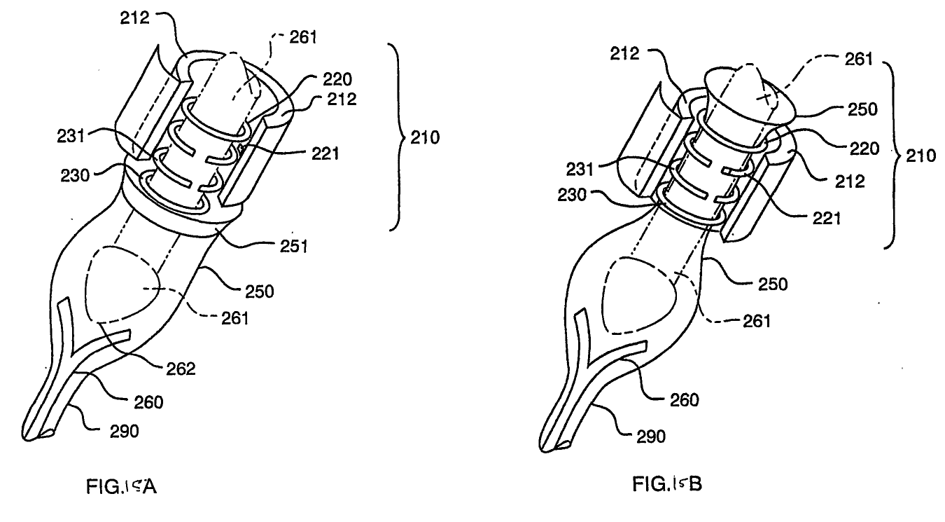

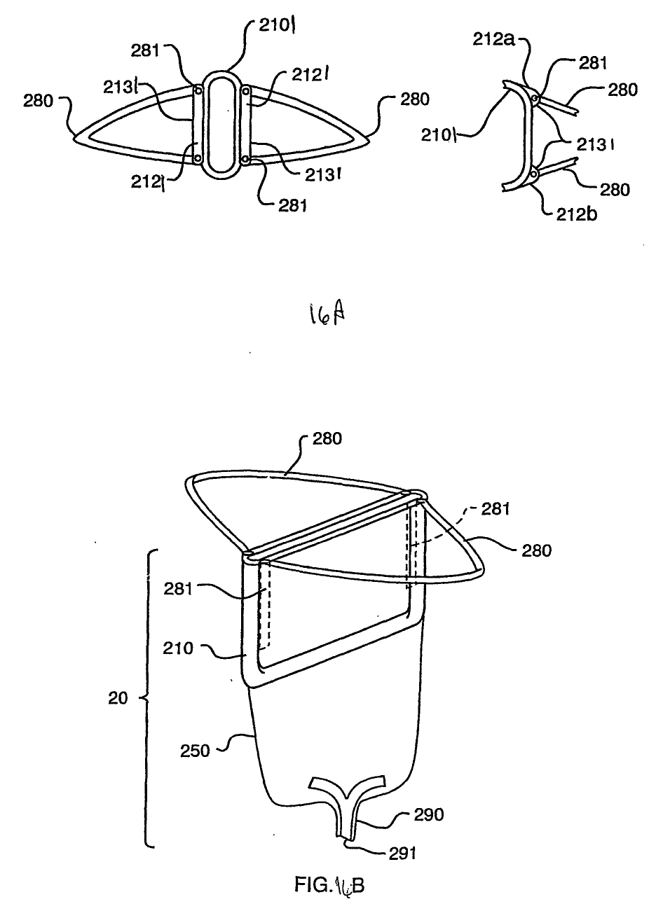

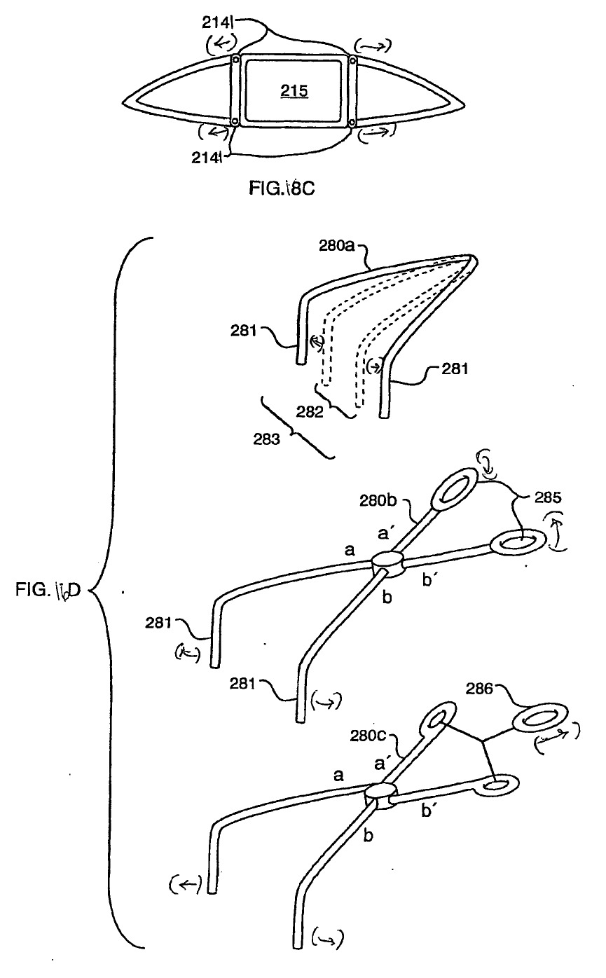

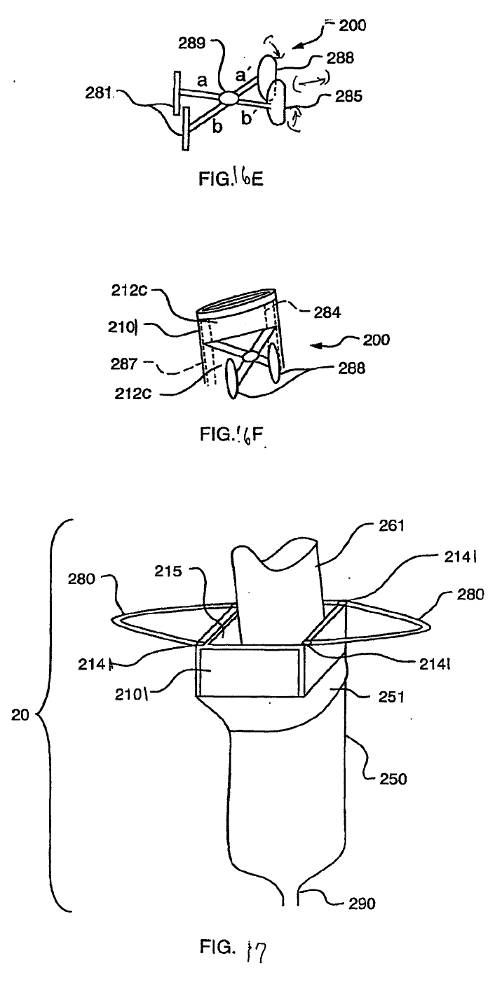

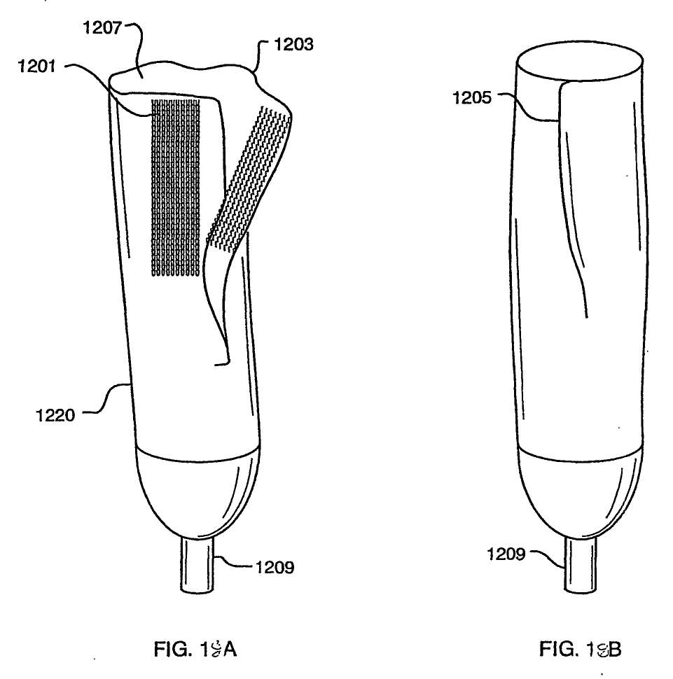

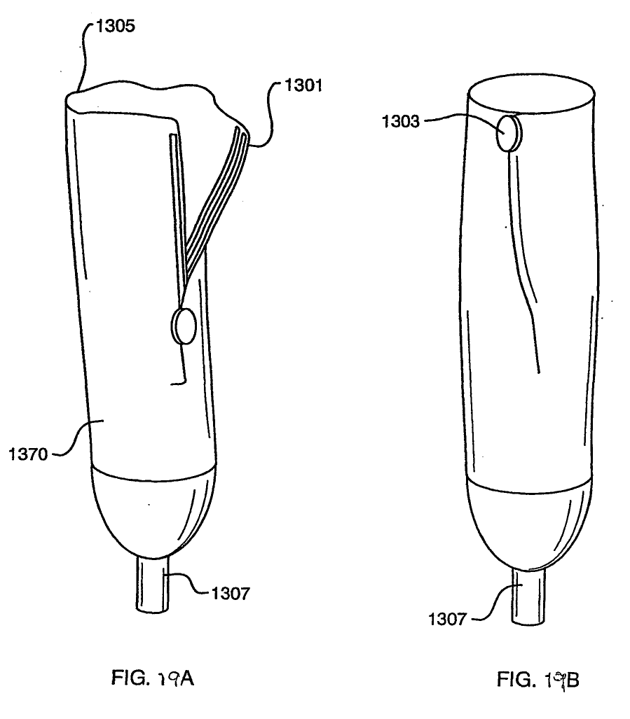

Referring now to FIG. 9A, a urine storage device 30 of the compartmental type comprises at least one each of urine distributor 341, liquid barrier wall 330 that has some regions of liquid permeability 331, and an absorbent material 350, all in contiguous fluid contact when liquid is conveyed along spacer 340. Components 341, 330, and 350 are contained within a structure formed by sealing a liquid-impervious back wall 320 and liquid-impervious front wall 310 along their entire peripheral edges 311 except in the vicinity of conveyance tube-storage device connector 60. Peripheral edges 311 are sealed using adhesive or heat-sealing bonding techniques that are well known in the art

Urine storage device 30 of the compartmental type may comprise one or more than one compartment that contains urine distributor 341, liquid permeable barrier wall 330, and absorbent materials 350 by forming walls and edges that partially enclose the compartment. In FIGS. 9A and 10, storage device 30 is shown with three compartments delineated by compartment-defining lines 360. In an embodiment with a single compartment, seal lines 360 will not be present.

As illustrated in FIG. 11, front wall 310 can be either cut large or have extra wall material added to it, said extra materials being folded in at their peripheral edges 312, to permit the compartment volume to expand when liquid absorption occurs.

It should be understood that there may be additional wall structures, which are exterior to the urine-impervious front wall 310 or back wall 320 of urine storage device 30, said exterior walls being added for various purposes such as to aid resistance to tearing or puncturing of said walls, to provide surface comfort for the wearer, or for decorative purposes.

Referring to FIGS. 9A, 9B and 9C, storage device barrier wall 330 is intended to regulate desired flow of urine through the barrier in a pattern that results in a more or less uniform wetting of absorbent array 350. The barrier wall 330 can be a thin urine-impervious film that is perforated with as few as one or an array of barrier wall holes 331 of sufficient size and number or be made of materials that are semi-permeable to urine. Urine distributor 341 is a capillary wicking material located between back wall 320 and barrier wall 330. Spacer 340, located within connector 41, extends to make contact with wicking distributor 341 to provide a continuous wicking path from the conveyance tube spacer 106 to storage devise distributor 341.

Absorbent material 350 is located in each of one or more separate compartments in urine storage device 30 in contact with the fluid passage points 331 or permeable surfaces of barrier wall 330 in order to facilitate uniform distribution of absorbed urine. Absorbent material 350 may be part of a physically stable structure, thus, forming an absorbing matrix, or may be present as un-aggregated solids within each compartment. Absorbent Material 350 may be materials that absorb aqueous fluids, such as: gel-forming resins (polyacrylamide, polyacrylic acid or its Na+ salt, polyacrylic acid grafted onto starch or its Na+ salt), cellulose or other natural or synthetic fibers that may themselves be partially or wholly coated with layers or fine particles of such gel-forming resins, felts or mats of fibers, inorganic absorbents (silica gel and calcium sulfate), and may be combinations of absorbents. It will be clear to those skilled in the art that there are a wide variety of absorbent materials that fall within the spirit of the invention.

Referring now to FIGS. 2, 7A, and 9A, for attachment of urine storage device 30 to the human body, the device can be folded around the contour of a limb, for example, the calf, with back wall 320 toward the calf, and secured in place by encircling the limb with leg straps 321 and attaching them to the opposite straps or to the front wall 310 using adhesive, hook-and-loop or other fastening means. Storage device 30 can likewise be attached to other contoured body regions such as the hip by using encircling the waist with somewhat longer straps, or can be attached to an article of user's clothing such as underpants. Storage device 30 can also be attached to fixtures or even furniture near the user, such as a chair or wheelchair, when the user will be remaining in that place for some time period. It should be clear that there are many such attachment positions that will be convenient for the range of potential users.

Systems of Devices for Collection Conveyance, and Storage of Urine

In hydraulically connected systems, liquid flows by gravity from a higher elevation to a lower elevation. In the presence of liquid absorbing materials, the physical and chemical affinity of the absorbent toward the liquid can counter to a limited extent the gravitational force and transport the liquid in an ascending path. In a connected capillary system, surface tension provides to a limited extend a driving force to cause the liquid to flow in a ascending path provided that the discharge point is at a lower elevation than the inlet. In a connected urine wicking system of the instant invention, the gravity, absorbent affinity and capillary action all play a part in influencing the direction and rate of flow of urine within these connected elements.

Referring to FIG. 1, each of storage device 30, conveyance tube 10, and urine collector 20 contains a wicking element throughout the working length of the lumen of the device. When the devices 10, 20, and 30 are serially connected to form system 50, as shown in FIG. 1, the respective wicking elements are brought into contiguous contact by the coupling of connections 60 and 70 to form a continuous wicking connection the collector and the storage device.

By using a material that is easily wetted by urine as the inner wall or body-contact surface of the collector 20, urine droplets will, upon contact with wettable surfaces, wet the wettable surfaces and spread across them. When wettable surfaces, including the collector surface and the skin of the uro-genital region, are themselves in contact with a wicking element, then the urine will be imbibed to the wicking system and conveyed to the storage device, leaving the surfaces essentially free of liquid urine.

Removal of the excess liquid urine from the urogenital skin and from surfaces in collector 20 and conveyance tube 10 also reduces the volume of nutrients and fluid urine available to the various microbiological organisms that can grow and flourish in stale, standing urine. These microorganisms are frequently the source of urine decomposition products (e.g. ammonia) that are detrimental to the moist skin.

Since the wick remains wetted with urine, the possibility exists that microorganisms may grow and thus become transferable back to the user. As a means to limit the growth of undesirable microorganisms in the urine within the wick, antibacterial materials may be applied to the wick substrate as surface coatings or treatments, or may be compounded into fibers, formed into similar wicking materials and attached in contiguous contact with the wicking spacer. There are commercially available antibacterial materials whose properties are appropriate for the human contact use and that are effective against microorganisms frequently found in the urogenital region.(e.g., Escherichia coli, Pseudomonas aeruginosa). One example of such a commercial antibacterial material is Surfacineâ, a silver-based antibacterial coating material from Surfacine Development Company, Tewksbury, Mass. To prevent growth of bacteria, wicking elements within collection and conveyance devices may be treated with antibacterial substances.

In order to enable effective connection of novel urine collection device 20 to existent art tubes for conveying urine, and referring now to FIG. 12C, connection 290 attached to conduction tube 250 may be connected through adapter fitting 24 that will cause wicking spacers 260 and 23 to be located in a region within the connection that will not interfere with gravitational flow of urine into connected existent art urine tube 10EX.

Conveyance tube 10, also can be used independent of system 50 to perform the function of conveyance in combination with one or more of the existent other devices for collecting urine (e.g., condom catheters) and storing urine (e.g., leg bags), none of which are equipped with means for the novel wicking transport of this invention. Connection of device 10 for such independent use can be accomplished by replacing connecting devices used in system connections 60 and 70 with special connecting devices that are dimensioned to connect directly with existent devices to facilitate formation and transport of drops or streams of liquid urine 9 that can be handled by the existent devices. FIGS. 12A and 12B show different configurations for conveyance tube inlet 11 connecting with connector 290 from either a novel or an existent art collection device. In FIG. 12D, an existent art leg bag collection device 30EX is shown connected to novel conveyance tube 10 (which is itself connected to novel urine collector 20) via connector 25 and receiving drops of liquid urine 9 that are being formed at a drip point that is part of connector 25 and that is physically located lower than the urine source.

Referring now to FIGS. 13A-C, a continuous wicking path facilitates siphoning drainage over high point 120 as shown in FIG. 13A, enables storage of urine in storage device 30 that are higher than collection device 20 as shown in FIG. 13B, and, as shown in FIG. 13C, collects and removes to storage isolated residual pools and drops 9 of urine that might otherwise remain in urine collection device 20 or in urine conveyance tube 10, and cause irritation and injury to the skin of penis 261 from the moisture and decomposition products of the urine.

Referring now to FIG. 14, a variation of an illustrative embodiment is shown in which conveyance tube 10 is delivering urine to multiple storage devices 30. In this embodiment, conveyance tube 10 is split to present urine to more that one storage device 30 located at different physical positions. Multiple storage devices 30 may be of different shapes and sizes from each other, and may be in different physical locations from each other, such as one attached to the user's body and one attached to or placed on a nearby piece of furniture or fixture. Such multiple connections can provide a user with convenient means to move to multiple locations and re-connect to a larger size storage device.

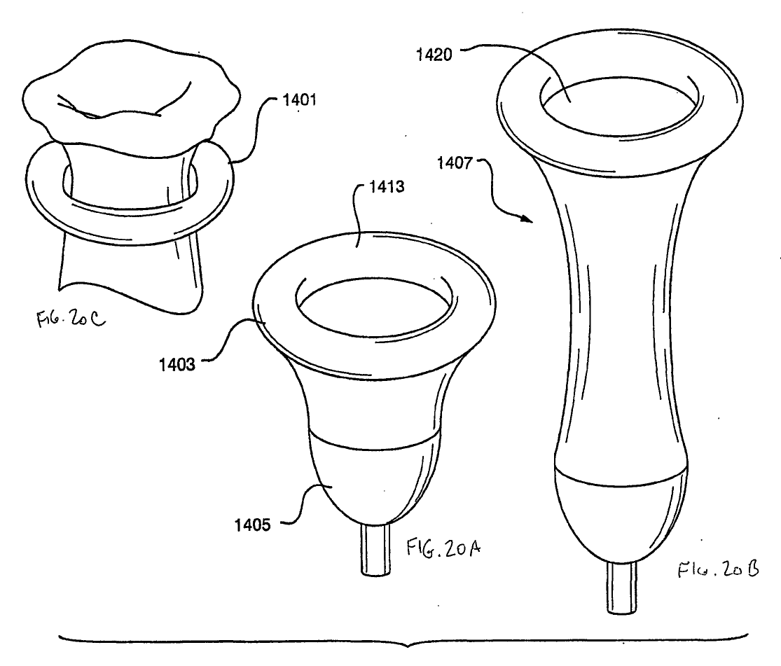

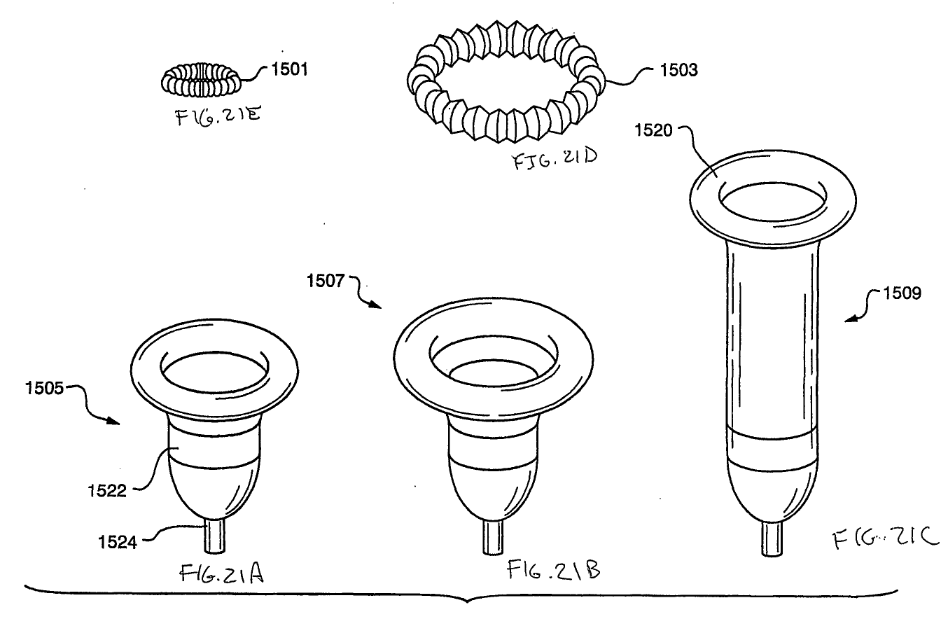

Referring now to FIGS. 18A-B, tubular sheath 1220 is shown fully extended with cavity 1207 ready to accept the penis for urine collection, and connection 1209 ready to receive collected urine. Sheath 1220 is open at slit 1203, which is disposed with multiple-hook-and-loop fasteners 1201. When sheath 1220 is in place on the penis, hook-and-loop fasteners 1201 are used to close slit 1203. Similarly, and now referring to FIGS. 19A-B, sheath 1320 connected to conveyance device 1307 is disposed with slit 1301 which is brought together by zip-lock-like fasteners 1303 around the penis which sits in cavity 1305.

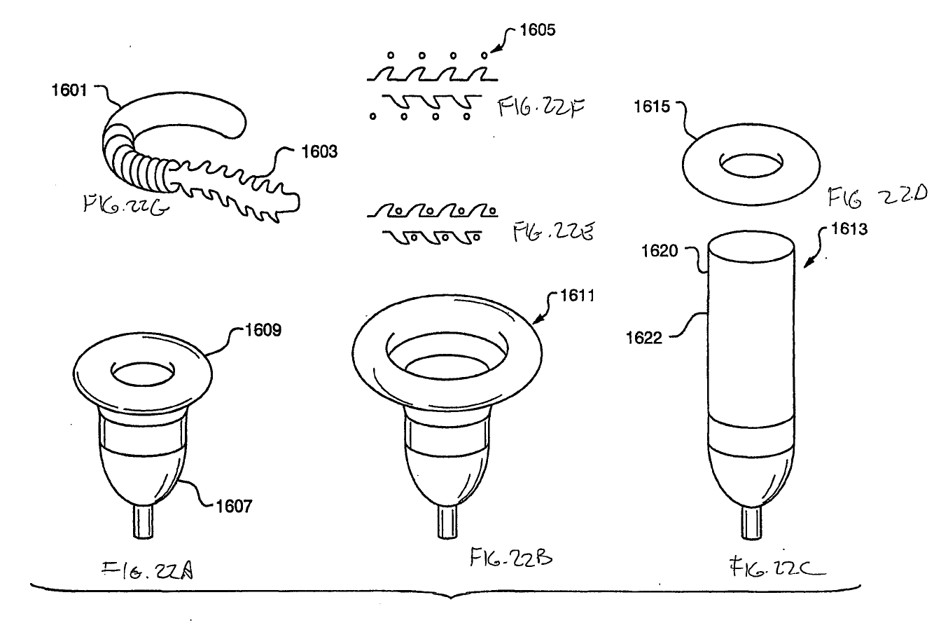

Referring now to FIGS. 20A-C, 21A-E, and 22A-G, devices that enable users to apply sheaths 1407, 1509, and 1613 onto a penis are disclosed. Elastic tubing 1401 is fixedly connected to sheath top 1403 in preparation for rolling sheath 1407 downwards longitudinally from penis entry cavity 1420 to conveyance device connector 1405. The user unrolls rolled sheath 1413 upwards along the shaft of the penis until sheath 1407 is fully extended. Elastic tubing 1401 helps sheath 1407 to grip and to maintain its position on the penis.

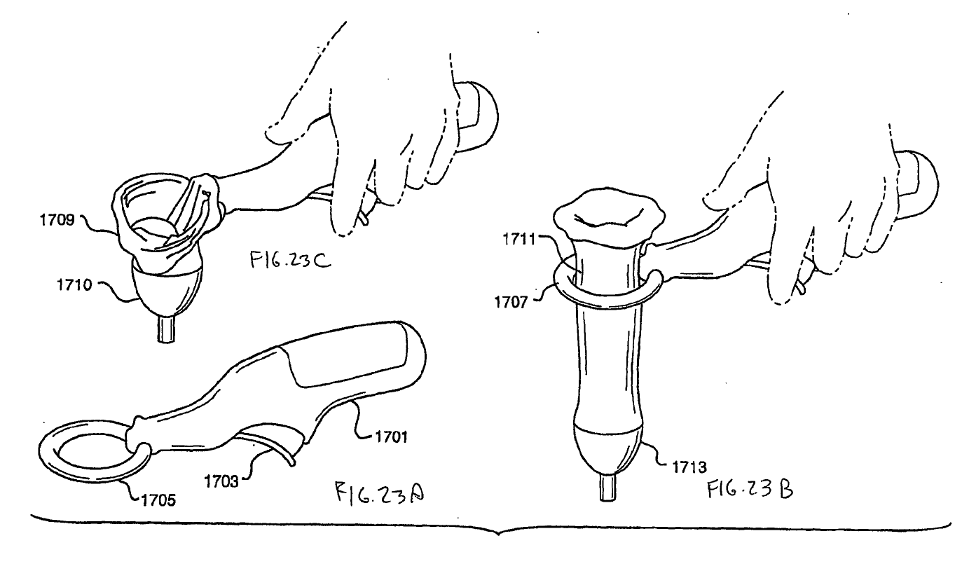

Referring now to FIGS. 20A-C and 21A-E, for sheaths that can maintain their positions through their own elastic or other frictional properties without the aid of elastic tubing 1401, hook-and-loop fasteners 1201, or zip-lock-type fasteners 1303, reversibly expandable rings such as rigid plastic bellows 1501 can be employed. Bellows ring 1501 is removably attached in its unexpanded state to sheath top 1520, and co-rolled sheath with attached bellows ring 1505 can be rolled down towards conveyance device connection 1524. In use, the user pulls rolled sheath and bellows ring 1505 to expand the rolled diameter 1507, and unrolls the expanded diameter sheath 1509 upwards longitudinally along the penis shaft. As sheath 1509 unrolls and is removed from close proximity to expanded bellows ring 1503, sheath 1509 shrinks in diameter and grips the penis. When sheath 1509 is nearly completely extended, expanded bellows ring 1503 and the remaining sheath can be compressed and unrolled to a point where compressed bellows ring 1501 can be detached from the sheath Detached bellows ring 1501 can then be pulled to expanded state 1503, removed from the penis, and discarded. Sheath 1509 can maintain its position on the penis shaft through its own elasticity. Along the same lines, and referring now to FIGS. 22A-G, spring coil ring 1615 comprising spring coil 1601 and contained toothed plastic strap 1603, is removably attached to the cavity perimeter 1620 of sheath 1622 and rolled down longitudinally, sheath 1622 and spring ring 1615 together, towards conveyance connector 1607. Spring ring 1615 is completely enclosed by sheath 1622. Before use, the user pulls on the co-rolled sheath and ring 1609 to expand the diameter of the co-rolled ring and sheath 1611, places the open end over the penis, and unrolls sheath 1611 along the shaft of the penis away from the penis tip until sheath 1622 is fully extended 1613. As it unrolls and is removed from close proximity to the expanded spring ring, elastic sheath 1622 shrinks in diameter and grips the penis. When sheath 1622 is completely extended, spring ring 1601 is detached from cavity perimeter 1620 of sheath 1622, which retracts elastically to grip the penis. Detached ring 1615 is removed from the penis and discarded.

Referring now to FIG. 23A-C, hand-held jaw ring expander 1701 for extending the sheath over the penis is disclosed. The user compresses lever 1703, which causes expansion ring 1705 to open In use, the end of sheath 1711 that is attached to conveyance device connector 1713 is inserted into the expansion ring 1705, in its inactivated compressed state, and penis entry end 1720 of sheath 1711 is draped 1709 over ring 1705. Then device lever 1703 is compressed, causing expansion ring 1705 and surrounding draped sheath 1709 to increase in diameter. Expanded ring 1705 and sheath 1711 are then moved longitudinally up the shaft of the penis. When sheath 1711 is nearly fully extended, device lever 1703 is released, allowing ring 1705 and expanded sheath 1711 to contract. Any remaining sheath 1711 is then unrolled onto the penis leaving sheath 1711 self-secured on the penis through its own elasticity. Device lever 1703 is again compressed, which enlarges ring 1703 and allows it to be removed from the penis.

It is thought that the present invention and many of its attendant advantages are understood from the foregoing description. It will be apparent that various changes may be made in the form, construction, and arrangement of the parts thereof without departing from the spirit and scope of the invention or sacrificing all of its material advantages, the form hereinbefore described being merely an illustrative embodiment thereof.

1. An improved urine management system for humans comprising
a collection device for collecting urine, the collection device having a urine entrance and a urine exit, the collection device forming a collection fluid path between the urine entrance and the urine exit, the collection fluid path containing contiguously connected fluid-wicking elements;
a conveyance tube for receiving urine from the collection device, the conveyance tube operably connected to the collection device at the urine exit, the conveyance tube having a first wicking connection with the collection device, the first wicking connection forming a collector-conveyance fluid path between the collection device and the conveyance tube, the collector-conveyance fluid path having continuous fluid connection with the collection fluid path, the conveyance tube having an interior, the interior forming a conveyance fluid path through the conveyance tube, the conveyance tube containing contiguously connected fluid-wicking elements; and
a storage container for receiving urine from the conveyance tube, the storage container operable connected to the conveyance tube, the storage container capable of storing urine, the storage container having a second wicking connection with the conveyance tube, the second wicking connection forming a conveyance-storage fluid path between the conveyance tube and the storage container, the conveyance-storage fluid path having continuous fluid connection, the conveyance-storage fluid path having contiguously connected fluid-wicking elements, wherein the collection fluid path, the collection-conveyance fluid path, the conveyance fluid path, and the conveyance-storage fluid path together form a system-wide continuous fluid path, the system-wide continuous fluid path having contiguously connected fluid-wicking elements.

2. The improved urine management system of claim 1 wherein the urine entrance further comprises an intake wall for moving trickle-type urine flow, urine residual, and urine pools along the collection fluid path towards the urine exit, said intake wall having contiguously connected fluid-wicking elements.

3. The improved urine management system of claim 1 wherein the system-wide continuous fluid path is capable of moving urine counter-gravitationally.

4. The improved urine management system of claim 1 further comprising a spacer within the system-wide continuous fluid path, the spacer preventing complete blockage of the system-wide continuous fluid path.

5. The improved urine management system of claim 1 wherein the storage container further comprises
a fluid distribution system for distributing urine substantially uniformly within the storage container; and
a fluid immobilizing system for converting distributed urine to immobilized form.

6. The improved urine management system of claim 1 wherein the storage container is disposable.

7. In a urine management system having a collection device, an improvement comprising
a conveyance tube for conveying urine from the collection device, the conveyance tube having an interior, the interior being occupied by a continuous wicking spacer;
a connector adapted to provide continuous fluid connection between the collection device and the continuous wicking spacer;
a storage container for receiving and storing urine from the conveyance tube, the storage container having continuous fluid connection with the conveyance tube, the storage container having contiguously connected fluid-wicking elements.

8. In a urine management system having a storage container, an improvement comprising [symbol] entrance and a urine exit, the collection device forming a collection fluid path between the entrance and the exit, the collection fluid path having contiguously connected fluid-wicking elements;
a conveyance tube for receiving urine from the collection device exit, the conveyance tube having a first wicking connection with the urine exit, the first wicking connection forming a collector-conveyance fluid path between the urine exit and the conveyance tube, the collector-conveyance fluid path having continuous fluid connection with the collection fluid path, the conveyance tube having an interior, the interior forming a conveyance fluid path through the conveyance tube, the conveyance fluid path having contiguously connected fluid-wicking elements, the conveyance fluid path having continuous fluid connection with the collection-conveyance fluid path; and
a connector adapted to provide a conveyance-storage fluid path between the conveyance tube and the storage container, the connector having contiguously connected fluid-wicking elements.

9. An improved human urine collection device comprising
an intake layer having a body contact surface and an opposing surface, the body contact surface receiving discharged urine and wicking discharged urine away from the body contact surface towards the opposing surface;
a wall layer having an inner surface and an outer surface, the inner surface forming a continuous connection with the opposing surface; and
a transport medium for receiving urine from the intake layer and transporting urine outside the outer surface, the transport medium located substantially between the body contact surface and the opposing surface.

10. The improved human urine collection device of claim 9 further comprising
a hydrophobic layer situated between the body contact surface and the opposing surface, the hydrophobic layer having an enabling surface, an opposing inhibiting surface, and a plurality of openings, the enabling surface situated substantially parallel with the body contact surface, the enabling surface allowing the one-way passage of urine [symbol] passage of urine towards the body contact surface through the plurality of openings.

11. The improved human urine collection device of claims 9 or 10 wherein the body contact surface is contoured to fit human female anatomy.

12. The improved human urine collection device of claim 9 wherein the body contact surface is contoured to fit human male anatomy.

13. An improved human urine collection device for females comprising
an intake layer having a body contact surface and an opposing surface, the body contact surface receiving discharged urine and wicking discharged urine away from the body contact surface towards the opposing surface;
a hydrophobic layer situated between the body contact surface and the opposing surface, the hydrophobic layer having an enabling surface and an opposing inhibiting surface, the hydrophobic layer having at least one opening, the enabling surface situated substantially parallel with the body contact surface, the enabling surface allowing the one-way passage of urine through the at least one opening, the opposing inhibiting surface inhibiting reverse passage of urine towards the body contact surface through the at least one opening;
a wall layer having an inner surface and an outer surface, the inner surface forming a continuous connection with the opposing surface; and
a transport medium for receiving urine from the intake layer and transporting urine outside the outer surface, the transport medium located substantially between the body contact surface and the opposing surface.

14. The human urine collection device for females of claim 13 wherein the body contact surface is conformable to fit human female anatomy.

15. The human urine collection device for females of claim 13 further comprising a [symbol] discharged urine.

16. An improved human urine collection device for males comprising
a conduction tube for receiving discharged urine from a discharge location and wicking discharged urine away from the discharge location, the conduction tube having tube walls and a conduction tube interior;
a retaining apparatus to hold the conduction tube in proximity to the discharge location; and
a lumen for collecting and transferring urine outside of the conduction tube, the lumen located within the conduction tube interior, the lumen having a lumen interior.

17. The human urine collection device for males of claim 16 wherein the conduction tube is conformable to fit human male anatomy.

18. The human urine collection device for males of claim 16 wherein the lumen further comprises a spacer

19. The human urine collection device for males of claim 16 wherein the retaining apparatus is selected from a group consisting of a compression tube, a fastener and an adhesive.

20. The human urine collection device for males of claim 16 further comprising a spacer located within the conduction tube interior, the spacer substantially preventing complete sealing contact of the conduction tube interior, the complete sealing contact preventing flow of urine within the conduction tube interior.

21. The human urine collection device for males of claim 16 wherein the conduction tube further comprises
a pouch-type shape;
a urine-impervious exterior;

[symbol]

22. An improved urine transport medium for conducting human urine from a urine collection device comprising
a collector extension, the extension having an interior, the interior being filled with a spacer and a wick, the collector extension having at least one open channel, the at least one open channel acting as a transitory reservoir for urine; and
an extension connector, the extension connector operably connecting the collection device with the collector extension, the extension connector having wicking connection with the urine collection device and the collector extension.

23. An improved urine conveyance device to conduct urine from a urine collection device to at least one urine storage container, the conveyance device comprising
a conduction tube having at least one wall, wherein one of at least one wall is urine-impervious, the conduction tube having an interior cavity within said at least one wall, the conduction tube having a first connecting end and a second connecting end, the first connecting end operably connecting the conduction tube with the urine collection device, the second connecting end operably connecting the conduction tube with the at least one urine storage device; and
a spacer disposed within the conduction tube, the spacer preventing the at least one wall from forming a complete seal, the complete seal preventing urine conduction within the conduction tube.

24. The urine conveyance device of claim 23 wherein the at least one wall is thin, flexible, and capable of conforming to various contours and shapes.

25. The urine conveyance device of claim 24 wherein the conduction tube is capable of becoming substantially flat when the interior cavity is empty.

26. An improved urine storage container comprising
an inlet connector for receiving urine from a urine source; [symbol] layer, the outer shell connected to the inlet connector, the cavity containing at least one compartment;
a urine distributor within the outer shell for uniformly distributing the received urine within the at least one compartment; and
a urine-immobilizing absorbent within the at least one compartment, the absorbent accepting urine from the distributor and converting it to non-liquid form.

27. The urine storage container of claim 26 wherein the urine distributor comprises urine-wicking material.

28. The urine storage container of claims 26 or 27 wherein the urine-immobilizing absorbent is selected from a group consisting of a matrix of non-woven fibers, one or more solid absorbent materials held in place by a structure, and one or more absorbent materials coated onto the cavity walls.

29. The urine storage container of claim 26 further comprising an attachment apparatus for maintaining the position of the storage container, the attachment apparatus connected to the outer shell.

30. The conveyance device of claims 23 and 24 wherein the spacer is capable of wicking urine counter-gravitationally and gravitationally away from the urine collection device, the spacer being further capable of preventing complete sealing contact of the interior cavity.
